(19) United States
(12) Patent Application Publication
(10) Pub. No.: US 2025/0381148 A1
(43) Pub. Date: Dec. 18, 2025

(54) PREPARATION OF ABUSE-DETERRENT PHARMACEUTICAL FORMULATIONS USING VAPOR PHASE DEPOSITION

(71) Applicant: Applied Materials, Inc., Santa Clara, CA (US)

(72) Inventors: Shrikant Swaminathan, Santa Clara, CA (US); David Masayuki ISHIKAWA, Mountain View, CA (US); Neha GUPTA, Mumbai (IN); Miaojun WANG, Santa Clara, CA (US); Balaji GANAPATHY, Maharashtra (IN); Fei WANG, Fremont, CA (US); Pravin K. NARWANKAR, Sunnyvale, CA (US)

(21) Appl. No.: 19/236,809

(22) Filed: Jun. 12, 2025

(30) Foreign Application Priority Data
Jun. 13, 2024 (IN) 202441045755

(51) Int. Cl. A61K 9/50; A61K 31/485; A61K 31/495; A61K 47/02; C23C 16/40; C23C 16/44; C23C 16/455

(52) U.S. Cl. CPC A61K 9/501; A61K 9/5089; A61K 31/485; A61K 31/495; A61K 47/02; C23C 16/403; C23C 16/4417; C23C 16/45527

(57) ABSTRACT

The disclosure is directed at an abuse-deterrent pharmaceutical composition comprising a coated antagonist particle, and the use thereof for preventing or minimizing the risk of opioid abuse and/or opioid toxicity from either intentional or unintentional tampering. Methods for manufacturing such an abuse-deterrent pharmaceutical composition are also provided.

### CROSS-REFERENCE TO RELATED APPLICATIONS

This application claims the benefit of priority to Indian Provisional Application No. 202441045755, filed on Jun. 13, 2024, the contents of which are hereby incorporated by reference.

### TECHNICAL FIELD

The present disclosure is in the field of abuse-deterrent pharmaceutical formulations, extended release pharmaceutical formulations, and extended release abuse-deterrent pharmaceutical formulations.

### BACKGROUND

Opioids, also called opioid agonists, are a class of drugs that exhibit opium-like or morphine-like properties. A major concern associated with the use of opioids is opioid abuse by non-medical users. Opioids may be ingested whole, crushed and ingested, crushed or vaporized and snorted or injected intravenously after attempted extraction of the active pharmaceutical ingredient. For instance, pentazocine (Talwin®), a synthetic opioid was crushed, extracted and injected intravenously by drug addicts.

Previous attempts to control the abuse potential associated with opioids include, for example, the combination of pentazocine and naloxone in tablets. The inclusion of naloxone is intended to curb a form of misuse of oral pentazocine, which occurs when the formulation is solubilized and injected.

There is a need for effective abuse-deterrent formulations wherein an antagonist that is conditionally release upon tampering. The benefits of the abuse-deterrent formulations are especially great in connection with oral formulations of strong opioid agonists (e.g., morphine, hydromorphone, oxycodone or hydrocodone), which provide valuable analgesics but are prone to being abused. This is particularly true for sustained-release opioid agonist products, which have a large dose of a desirable opioid agonist intended to be released over a period of time in each dosage unit. Drug abusers take such sustained release product and crush, grind, extract or otherwise damage the product so that the full contents of the formulation become available for immediate absorption.

### SUMMARY

This disclosure is related to abuse-deterrent formulations comprising (1) a coated antagonist particle comprising an antagonist-containing core, and (2) an coated drug particle. The coating can be applied by vapor phase deposition. The coated particles have a modified drug/antagonist release profile compared to the uncoated particles. Upon administration of the formulation to a subject, the antagonist is not substantially released into the blood stream of the subject and the efficacy of the drug is not substantially antagonized. However, tampering with the formulation, e.g., by crushing, grinding or wetting, alters the release profile of the antagonist relative to the release profile of the drug such that an altered formulation is created. When the altered formulation is administered to a subject, the antagonist counters the effect of the drug relative to when the unaltered formulation is administered to the subject. For example, in the altered formulation the antagonist is released more rapidly and/or to a greater extent relative to the unaltered formulation, for example, because the coating on the antagonist is damaged or because the coated antagonist drug particles are fractured or broken into smaller particles exposing uncoated surfaces.

In general, the uncoated antagonist particle is coated with an antagonist coating layer to create the coated antagonist particle, and the uncoated drug particle is coated with a drug coating layer to create the coated drug particle and the particles are combined to produce an abuse-deterrent formulation. The antagonist coating layer of the coated antagonist particles can be thicker and/or more resistant to erosion than the drug coating layer on the coated drug particles. Because of this, upon normal administration, the drug will be more readily released than the antagonist.

Both the antagonist coating layer and the drug coating layer can be multi-layer coatings that include two or more inorganic oxide (e.g., metal oxide or metalloid oxide) layers. For example, the antagonist coating layer can include an inner zinc oxide coating layer and an outer aluminum oxide coating layer. Both the antagonist coating layer and the drug coating layer can include a ternary compound coating layer, for example, an aluminum-zinc-oxide (AZO) coating. In the case where both the antagonist coating layer and the drug coating layer include an AZO coating, the Al/Zn ratio of the antagonist coating layer can be higher than the Al/Zn ratio of the drug coating layer.

In one aspect, the disclosure is related to an abuse-deterrent pharmaceutical composition, comprising:
(a) a coated antagonist particle comprising an antagonist-containing core comprising an antagonist, and an antagonist coating layer enclosing the antagonist-containing core; and
(b) an optionally coated drug particle,
wherein the antagonist coating layer comprises an inorganic oxide that comprises aluminum, zinc, silicon, and/or titanium.

In some embodiments, the drug particle comprises a drug-containing core comprising a drug and a drug coating layer enclosing the drug-containing core, wherein the drug coating layer comprises an inorganic oxide that comprises aluminum, zinc, silicon, and/or titanium selected from the group consisting of aluminum oxide, zinc oxide, silicon oxide and titanium oxide.

In some embodiments, the antagonist coating layer in the coated antagonist particle is different from the drug coating layer in the coated drug particle.

In some embodiments, the antagonist coating layer in the coated antagonist particle is thicker than the drug coating layer in the coated drug particle.

In some embodiments, the antagonist coating layer is 0.1 nm-120 nm thick.

In some embodiments, the antagonist coating layer is 5 nm-15 nm thick.

In some embodiments, the coated antagonist particle comprises 1-20% wt/wt inorganic oxide.

In some embodiments, the antagonist-containing core consists of an antagonist.

In some embodiments, the antagonist-containing core comprises an antagonist and one or more pharmaceutically acceptable excipients.

In some embodiments, the antagonist-containing core has a D50 on a volume average basis of 100 nm-30 micrometers.

In some embodiments, the antagonist-containing core has a median particle size, on a volume average basis, between 0.1 μm and 20 μm.

In some embodiments, the antagonist is selected from the group consisting of naltrexone, naloxone, nalmefene, cyclazacine, levallorphan, pharmaceutically acceptable salts thereof, and combinations thereof.

In some embodiments, the drug is an opioid.

In some embodiments, the inorganic oxide is selected from the group consisting of aluminum oxide, zinc oxide, silicon oxide and titanium oxide.

In some embodiments, the molar ratio between (1) the antagonist in the composition and (2) the drug in the composition is above 1.

In some embodiments, the molar ratio between (1) the antagonist in the composition and (2) the drug in the composition is above 2.

In some embodiments, both the antagonist coating layer and the drug coating layer comprise aluminum and zinc, and the aluminum/zinc ratio in the antagonist coating layer is higher than the aluminum/zinc ratio in the drug coating layer.

In some embodiments, the abuse-deterrent pharmaceutical composition comprises a dry mix of the coated antagonist particle and the optionally coated drug particle.

In some embodiments, the abuse-deterrent pharmaceutical composition comprises a pharmaceutically acceptable excipient or carrier.

In some embodiments, the antagonist negates the intended effect of the drug or produces an unpleasant or punishing stimulus or effect.

In some embodiments, the coated antagonist particle has a slower release rate comparing to an uncoated antagonist-containing core.

In some embodiments, the release rate of the coated antagonist particle is at least 100% slower than that of the an uncoated antagonist-containing core.

In some embodiments, the coated antagonist particle has an improved flowability comparing to an uncoated antagonist-containing core.

In some embodiments, upon tampering (e.g., grinding), the coated antagonist particle releases the antagonist at a faster rate.

In some embodiments, the tampering involves crushing or grinding.

In one aspect, the disclosure is related to a method of preparing an abuse-deterrent pharmaceutical composition, the method comprising the sequential steps of:
(a) loading particles comprising an antagonist into a chamber of a reactor;
(b1) applying a vaporous or gaseous precursor to the particles in the reactor by pulsing the vaporous or gaseous aluminum precursor into the reactor;
(b2) performing one or more pump-purge cycles using an inert gas;
(b3) applying a vaporous or gaseous oxidant to the particles in the reactor by pulsing the oxidant into the reactor;
(b4) performing one or more pump-purge cycles using an inert gas;
(c) repeating steps (b1)-(b4) at least once to create coated antagonist particles; and
(d) mixing the coated antagonist particles with coated drug particles.

In some embodiments, each pump-purge cycle comprises flowing the inert gas into the reactor chamber to reach a desired pressure and after a delay time pumping the inert gas out of the reactor until the pressure of the inert gas is below 1 torr.

In some embodiments, the coated drug particles are prepared by a method comprising the sequential steps of:
(A) loading particles comprising a drug-containing core comprising a drug into a chamber of a reactor;
(B1) applying a vaporous or gaseous precursor to the particles in the reactor by pulsing the vaporous or gaseous aluminum precursor into the reactor;
(B2) performing one or more pump-purge cycles using an inert gas;
(B3) applying a vaporous or gaseous oxidant to the particles in the reactor by pulsing the oxidant into the reactor;
(B4) performing one or more pump-purge cycles using an inert gas;
(C) repeating steps (B1)-(B4) at least once to create the coated drug particles.

In some embodiments, the resulting abuse-deterrent pharmaceutical composition comprises coated antagonist particles comprising an antagonist-containing core comprising an antagonist, and an antagonist coating layer enclosing the antagonist-containing core, wherein the antagonist coating layer is conformal, pinhole-free and comprises an inorganic oxide that comprises aluminum, zinc, silicon, and/or titanium.

In some embodiments, the resulting abuse-deterrent pharmaceutical composition comprises (1) coated antagonist particles consisting of an antagonist-containing core comprising an antagonist, and an antagonist coating layer enclosing the antagonist-containing core; and (2) coated drug particles consisting of a drug-containing core comprising a drug, and a drug coating layer enclosing the drug-containing core.

In some embodiments, the coated drug particles comprise a drug-containing core comprising a drug, and a drug coating layer enclosing the drug-containing core, wherein the drug coating layer is conformal, pinhole-free and comprises an inorganic oxide that comprises aluminum, zinc, silicon, and/or titanium.

In some embodiments, steps (b1)-(b4) take place at a temperature between 25° C. and 60° C.

In some embodiments, step (a) further comprises agitating the antagonist-containing particles.

In some embodiments, the precursor is an aluminum oxide precursor.

In some embodiments, the precursor is trimethylaluminium (TMA).

In some embodiments, the oxidant is water.

In some embodiments, the antagonist coating layer constitutes 1-20% wt/wt of the coated antagonist particles.

In some embodiments, the antagonist coating layer has a thickness in the range of 0.1 nm to 120 nm.

In some embodiments, the antagonist coating layer has a thickness in the range of 5 nm to 15 nm.

In some embodiments, the coated antagonist particles have a slower drug release rate comparing to uncoated antagonist particles.

In some embodiments, the antagonist is not degraded during the coating process.

In one aspect, the disclosure is related to a method of preparing an abuse-deterrent pharmaceutical composition, the method comprising the sequential steps of:

(a) loading particles comprising an antagonist into a chamber of a reactor;
(b) performing a first number of first cycles, wherein each first cycle comprises steps (b1)-(b4):
(b1) applying a vaporous or gaseous first inorganic oxide precursor to the particles in the reactor by pulsing the vaporous or gaseous first inorganic oxide precursor into the reactor;
(b2) performing one or more pump-purge cycles of the reactor using an inert gas;
(b3) applying a vaporous or gaseous oxidant to the particles in the reactor by pulsing the oxidant into the reactor;
(b4) performing one or more pump-purge cycles of the reactor using an inert gas;
   (c) performing a second number of second cycles, wherein each second cycle comprises steps (c1)-(c4):
(c1) applying a vaporous or gaseous second inorganic oxide precursor to the particles in the reactor by pulsing the vaporous or gaseous second inorganic oxide precursor into the reactor;
(c2) performing one or more pump-purge cycles of the reactor using an inert gas;
(c3) applying a vaporous or gaseous oxidant to the particles in the reactor by pulsing the oxidant into the reactor;
(c4) performing one or more pump-purge cycles of the reactor using an inert gas; and
   (d) repeating steps (b)-(c) at least once to create coated antagonist particles; and
   (e) mixing the coated antagonist particles with coated drug particles, wherein, the first inorganic oxide precursor and second inorganic oxide precursor are different and the first number is an integer selected from 1-10, and the second number is an integer selected from 1-10.

In some embodiments, the uncoated antagonist particles have a median particle size, on a volume average basis between 0.1 μm and 1000 μm.

In some embodiments, the coated drug particles are prepared by a method comprising the sequential steps of:

(A) loading a drug-containing core comprising a drug into a chamber of a reactor;
(B) performing a first number of first cycles, wherein each first cycle comprises steps (B1)-(B4):
(B1) applying a vaporous or gaseous first inorganic oxide precursor to the particles in the reactor by pulsing the vaporous or gaseous first inorganic oxide precursor into the reactor;
(B2) performing one or more pump-purge cycles of the reactor using an inert gas;
(B3) applying a vaporous or gaseous oxidant to the particles in the reactor by pulsing the oxidant into the reactor;
(B4) performing one or more pump-purge cycles of the reactor using an inert gas; (C) performing a second number of second cycles, wherein each second cycle comprises steps (C1)-(C4):
(C1) applying a vaporous or gaseous second inorganic oxide precursor to the particles in the reactor by pulsing the vaporous or gaseous second inorganic oxide precursor into the reactor;
(C2) performing one or more pump-purge cycles of the reactor using an inert gas;
(C3) applying a vaporous or gaseous oxidant to the particles in the reactor by pulsing the oxidant into the reactor;
(C4) performing one or more pump-purge cycles of the reactor using an inert gas; and
   (D) repeating steps (B)-(C) at least once to create coated drug particles, wherein, the first inorganic oxide precursor and second inorganic oxide precursor are different and the first number is an integer selected from 1-10, and the second number is an integer selected from 1-10.

In some embodiments, the resulting abuse-deterrent pharmaceutical composition comprises coated antagonist particles consisting of an antagonist-containing core comprising an antagonist, and an antagonist coating layer enclosing the antagonist-containing core, wherein the antagonist coating layer is conformal, pinhole-free and comprises an inorganic oxide that comprises aluminum, zinc, silicon, and/or titanium.

In some embodiments, the resulting abuse-deterrent pharmaceutical composition comprises (1) coated antagonist particles consisting of an antagonist-containing core comprising an antagonist, and an antagonist coating layer enclosing the antagonist-containing core; and (2) coated drug particles consisting of a drug-containing core comprising a drug, and a drug coating layer enclosing the drug-containing core.

In some embodiments, the coated drug particles comprise a drug-containing core comprising a drug, and a drug coating layer enclosing the drug-containing core, wherein the drug coating layer is conformal, pinhole-free and comprises an inorganic oxide that comprises aluminum, zinc, silicon, and/or titanium.

In some embodiments, the first inorganic oxide precursor is an aluminum oxide precursor and the second inorganic oxide precursor is a zinc oxide precursor.

In some embodiments, the first inorganic oxide precursor is a zinc precursor and the second inorganic oxide precursor is an aluminum oxide precursor.

In some embodiments, the aluminum oxide precursor is trimethylaluminum (TMA).

In some embodiments, the zinc oxide precursor is diethylzinc (DEZ).

In some embodiments, the coating constitutes 1-20% wt/wt of the coated particles.

In some embodiments, each of the first and second inorganic oxide precursor is selected from DEZ and TMA and either: a) the first inorganic oxide precursor is TMA and the second inorganic oxide precursor is DEZ; or b) the first inorganic oxide precursor is DEZ and the second inorganic oxide precursor is TMA.

In some embodiments, the first number is 1 or 2 and the second number is an integer between 1 and 10.

In some embodiments, the first number is 1.

In some embodiments, the second number is 2, 3, 4 or 5.

In some embodiments, the second number is 3.

In some embodiments, the second number is 4.

In some embodiments, steps (b)-(c) occur 1-40 times.

In some embodiments, each of steps (b1), (b3), (c1) and (c3) comprises: (i) introducing the vaporous or gaseous inorganic oxide precursor into the chamber, (ii) allowing a holding time to pass, and (iii) pumping the vaporous or gaseous inorganic oxide precursor of the chamber; and repeating steps (i)-(ii) at least once.

In some embodiments, some or all of the residual vaporous or gaseous first inorganic oxide precursor is pumped out of the reactor prior to step (b3).

In some embodiments, some or all of the residual vaporous or gaseous oxidant is pumped out of the reactor prior to step (c).

In some embodiments, some or all of the residual vaporous or gaseous second inorganic oxide precursor is pumped out of the reactor prior to step (c3).

In some embodiments, the first cycles and second cycles take place at a temperature between 25° C. and 60° C.

In some embodiments, the oxidant in step (b3) is water.

In some embodiments, the oxidant in step (c3) is water.

In some embodiments, step (a) further comprises agitating the particles.

In some embodiments, each pump-purge cycle comprises flowing the inert gas into the reactor chamber to a desired pressure and after a delay time pumping the inert gas out of the reactor until the pressure of the inert gas is below 1 torr and repeating the steps of flowing the inert gas into the reactor chamber to a desired pressure and after a delay time pumping the inert gas out of the reactor until the pressure of the inert gas is below 1 torr.

In some embodiments, the inorganic oxide coating is a ternary compound.

In some embodiments, the ternary compound is composed of aluminum, zinc and oxygen.

In some embodiments, the method comprises agitating the particles in the reactor throughout steps (a)-(d).

In some embodiments, the particles are not removed from the reactor during steps (a)-(d).

In some embodiments, the thickness of the antagonist coating layer is 0.1-120 nm, 10-120 nm, 30-120 nm, or 50-120 nm.

In some embodiments, the antagonist coating layer has a thickness in the range of 0.1 nm to 120 nm.

In some embodiments, the antagonist coating layer has a thickness in the range of 5 nm to 15 nm.

In some embodiments, the coated antagonist particle have a slower drug release rate comparing to uncoated antagonist particles.

In some embodiments, the antagonist is not degraded during the coating process.

In one aspect, the disclosure is related to an abuse-deterrent composition prepared by the method described herein.

In some embodiments, the drug is an opioid, and when the particles are administered in their native state (without crushing), the plasma level of the antagonist is insufficient to blunt the effects of the opioid.

In some embodiments, the drug is an opioid, and the when the particles are crushed and administered, the plasma level of the antagonist is sufficient to blunt the effects of the opioid.

In some embodiments, the drug is an opioid, and the when the particles are tampered by solvent dissolution (e.g., using an acidic solution such as lemonade or vinegar) and administered, the plasma level of the antagonist is sufficient to blunt the effects of the opioid.

In some embodiments, upon tampering (e.g., grinding), the dissolution rate of the coated antagonist particle is increased.

The coating process can be performed at a low process temperature, e.g., below 80° C., e.g., at or below 50° C., at or below 35° C., or at or below 25° C. In some embodiments, the operating temperature is 50° C. In some embodiments, the operating temperature is above 5° C., above 10° C., above 15° C., above 20° C., above 25° C., above 30° C., above 35° C., above 40° C., above 45° C., above 50° C., above 56° C., above 60° C., above 65° C., above 70° C., above 75° C., or above 80° C. (e.g. 20° C. to 80° C.). In some embodiments, the operating temperature is below 20° C., below 25° C., below 30° C., below 35° C., below 40° C., below 45° C., below 50° C., below 56° C., below 60° C., below 65° C., below 70° C., below 75° C., or below 80° C. In particular, the particles can remain or be maintained at such temperatures during all of the coating steps (e.g., inorganic precursor steps, oxidant steps, pump-purge steps). This can be achieved by having the oxidant gas, precursor gas and inert gas be injected into the chamber at such temperatures during the respective cycles. In addition, physical components of the chamber can remain or be maintained at such temperatures, e.g., using a cooling system, e.g., a thermoelectric cooler, if necessary.

In some embodiments, the amount of inorganic component constitutes more than 0.1%, more than 0.2%, more than 0.3%, more than 0.4%, more than 0.5%, more than 0.6%, more than 0.7%, more than 0.8%, more than 0.9%, more than 1%, more than 1.2%, more than 1.4%, more than 1.6%, more than 1.8%, more than 2%, more than 2.2%, more than 2.4%, more than 2.6%, more than 2.8%, more than 3%, more than 3.2%, more than 3.4%, more than 3.6%, more than 3.8%, more than 4%, more than 4.2%, more than 4.4%, more than 4.6%, more than 4.8%, more than 5%, more than 6%, more than 7%, more than 8%, more than 9%, more than 10%, more than 12%, more than 14%, more than 16%, more than 18%, or more than 20% wt/wt of the coated particles.

In some embodiments, weight percent of inorganic oxides in the coated particles is less than 0.1%, less than 0.2%, less than 0.3%, less than 0.4%, less than 0.5%, less than 0.6%, less than 0.7%, less than 0.8%, less than 0.9%, less than 1%, less than 1.2%, less than 1.4%, less than 1.6%, less than 1.8%, less than 2%, less than 2.2%, less than 2.4%, less than 2.6%, less than 2.8%, less than 3%, less than 3.2%, less than 3.4%, less than 3.6%, less than 3.8%, less than 4%, less than 4.2%, less than 4.4%, less than 4.6%, less than 4.8%, less than 5%, less than 6%, less than 7%, less than 8%, less than 9%, less than 10%, less than 12%, less than 14%, less than 16%, less than 18%, or less than 20% wt/wt of the coated particles. In some embodiments, the weight percent of inorganic oxides in the coated particles is 0.1%-20%, 0.5%-10%, 1%-10%, 1%-5%, 2%-5%, 1%-4%, 1%-3%, or 2%-4% wt/wt of the coated particles. In some embodiments, the amount of inorganic component constitutes about 1%-20% wt/wt of the coated particles.

In some embodiments, the coated particles have a reduced dissolution rate compared to uncoated particles. In some embodiments, the dissolution rate of the coated particles is at least more than 5%, more than 10%, more than 20%, more than 30%, more than 40%, more than 50%, more than 60%, more than 70%, more than 80%, more than 90%, more than 100%, more than 110%, more than 120%, more than 130%, more than 140%, more than 150%, more than 200%, more than 300%, more than 400%, more than 500%, or more than 600%, lower than the dissolution of uncoated particles. In some embodiments, uncoated particles exhibited an immediate release profile.

In some embodiments, the coating layer prevents the release of the antagonist unless the formulation is tampered (e.g., by crushing or grinding). In some embodiments, the coating layer prevents the release of the antagonist in physiological conditions (e.g., pH 7.4). In some embodiments, the coating layer prevents the release of the antagonist but does not prevent the release of the drug from the composition. In some embodiments, when the formulation is tampered (e.g., by crushing or grinding), the antagonist is released from the formulation. In some embodiments, the antagonist is conditionally released upon tampering of the formulation. In some embodiments, the release rate of the antagonist is significantly increased upon tampering.

In some embodiments, the coated particles have 30-90% antagonist/drug release in 30 minutes. In some embodiments, the coated particles have a slow antagonist/drug release rate. In some embodiments, the coated particles are dispersed in PBS.

In some embodiments, the coated particles have more than 10%, more than 20%, more than 30%, more than 40%, more than 50%, more than 60%, more than 70%, more than 80%, more than 90%, more than 95% antagonist/drug release in 30 minutes. In some embodiments, the coated particles have less than 10%, less than 20%, less than 30%, less than 40%, less than 50%, less than 60%, less than 70%, less than 80%, less than 90%, less than 95% antagonist/drug release in 30 minutes. In some embodiments, the coated particles have 100% antagonist/drug release in 30 minutes.

In some embodiments, comparing to uncoated particles, the coated particles have at least more than 10%, more than 20%, more than 30%, more than 40%, more than 50%, more than 60%, more than 70%, more than 80%, more than 90%, more than 95% slower antagonist/drug release.

In some embodiments, the uncoated particles have an immediate release (e.g., about 90-100% antagonist/drug release in 30 minutes). In some embodiments, the coated particles have a controlled release (e.g., about 1-10% release in 30 minutes).

In some embodiments, the coated particles exhibit increased hydrophobicity comparing to the uncoated particles.

In some embodiments, the coated particles exhibit increased powder flowability (“FFc”) comparing to the uncoated particles.

In some embodiments, the coated particles exhibit increased bulk density comparing to the uncoated particles.

In one aspect, the disclosure relates to a method to coat particles using supercycles. A supercycle includes a first number of first cycles (e.g., TMA cycles) and a second number of second cycles (e.g., DEZ cycles). In some embodiments, the aluminum/zinc (Al/Zn) ratio in the coating can be adjusted by varying the number of first cycles (e.g., TMA cycles) and the number of second cycles (e.g., DEZ cycles). In some embodiments, the first number is an integer selected from 1-10, and the second number is an integer selected from 1-10. In some embodiments, the first number is 1, 2, 3, 4, 5, 6, 7, 8, 9, 10, 11, 12, 13, 14, 15, 16, 17, 18, 19, 20, 21, 22, 23, 24, 25, 30, 40, or 50. In some embodiments, the second number is 1, 2, 3, 4, 5, 6, 7, 8, 9, 10, 11, 12, 13, 14, 15, 16, 17, 18, 19, 20, 21, 22, 23, 24, 25, 30, 40, or 50. In some embodiments, the first number is 1 and the second number is an integer selected from 1-5. In some embodiments, the first number is 1 and the second number is 2. In some embodiments, the first number is 1 and the second number is 3. In some embodiments, the first number is 1 and the second number is 4.

In some embodiments, multiple supercycles are used to create a mixed (e.g., ternary compound) coating layer (AZO coating layer or ASO coating layer). In some embodiments, the number of supercycles is more than 1, more than 2, more than 3, more than 4, more than 5, more than 6, more than 7, more than 8, more than 9, more than 10, more than 15, more than 20, more than 25, more than 30, more than 35, more than 40, more than 45, more than 50, more than 60, more than 70, more than 80, more than 90, more than 100, more than 110, more than 120, more than 130, more than 140, or more than 150. In some embodiments, the number of supercycles is less than 2, less than 3, less than 4, less than 5, less than 6, less than 7, less than 8, less than 9, less than 10, less than 15, less than 20, less than 25, less than 30, less than 35, less than 40, less than 45, less than 50, less than 60, less than 70, less than 80, less than 90, less than 100, less than 110, less than 120, less than 130, less than 140, or less than 150. In some embodiments, the number of supercycles is 5-50, 5-25, or 5-10.

In some embodiments, multiple first supercycles with a first Al/Zn ratio are followed by multiple second supercycles with a second Al/Zn ratio to deposit an AZO coating with two or more different Al/Zn ratios or a gradient Al/Zn ratio.

In some embodiments, multiple supercycles are used to create a mixed aluminum and silicon oxide coating layer (ASO coating layer). In some embodiments, multiple first supercycles with a first Al/Si ratio are followed by multiple second supercycles with a second Al/Si ratio to deposit an ASO coating with two or more different Al/Si ratios or a gradient Al/Si ratio.

In some embodiments, 5-10 supercycles are performed wherein the first number is 1 and the second number is 3. In some embodiments, 5-25 additional supercycles are performed wherein the first number is 1 and the second number is 2.

In some embodiments, 5-10 additional supercycles are performed wherein the first number is 1 and the second number is 4. In some embodiments, 5-25 additional supercycles are performed wherein the first number is 1 and the second number is 3.

In some embodiments there are multiple different layers. For example an AZO layer can be combined with AlOx layer and/or ZnOx layer to provide various different coating structures. The AZO layer can be an inner layer and the AlOx or ZnOx layer can be an outer layer. In some embodiments, exemplary coating structures include an AlOx layer and an AZO layer or an ZnOx layer and an AZO layer.

In some embodiments, the coated particles contain a mixture of different coating layers. For example, an ASO layer can be combined with AlOx layer and/or SiOx layer to provide various different coating structures. In some embodiments, the ASO layer can be an inner layer and the AlOx or SiOx layer can be an outer layer. In some embodiments, exemplary coating structures include an AlOx layer and an ASO layer or an SiOx layer and an ASO layer.

In some embodiments, the aluminum/zinc (Al/Zn) ratio (wt/wt) in the coated particles is more than 0.01, more than 0.02, more than 0.03, more than 0.04, more than 0.05, more than 0.06, more than 0.07, more than 0.08, more than 0.09, more than 0.1, more than 0.11, more than 0.12, more than 0.13, more than 0.14, more than 0.15, more than 0.2, more than 0.25,more than 0.3, more than 0.35, more than 0.4, more than 0.45, more than 0.5, more than 0.6, more than 0.7, more than 0.9, or more than 0.9. In some embodiments, the aluminum/zinc (Al/Zn) ratio in the coated particles is less than 0.01, less than 0.02, less than 0.03, less than 0.04, less than 0.05, less than 0.06, less than 0.07, less than 0.08, less than 0.09, less than 0.1, less than 0.11, less than 0.12, less than 0.13, less than 0.14, less than 0.15, less than 0.2, less than 0.25, less than 0.3, less than 0.35, less than 0.4, less than 0.45, less than 0.5, less than 0.6, less than 0.7, less than 0.9, or less than 0.9. In some embodiments, the Al/Zn ratio in the coated particles is 0.1-0.5, 0.1-0.4, 0.1-0.3, 0.15-0.3 or 0.15-0.35.

In some embodiments, the entirety of the coating has a thickness in the range of 0.1 nm to 100 nm, 0.1 nm to 50 nm, 0.1 nm to 10 nm, 0.1 to 5 nm, 1 nm to 50 nm, 1 nm to 10 nm, or 1 nm to 5 nm. In some embodiments, the entirety of the coating has a thickness of more than 0.1 nm, more than 0.2 nm, more than 0.3 nm, more than 0.4 nm, more than 0.5 nm, more than 0.6 nm, more than 0.7 nm, more than 0.8 nm, more than 0.9 nm, more than 1 nm, more than 2 nm, more than 3 nm, more than 4 nm, more than 5 nm, more than 6 nm, more than 7 nm, more than 8 nm, more than 9 nm, more than 10 nm, more than 15 nm, more than 20 nm, more than 30 nm, more than 40 nm, more than 50 nm, or more than 100 nm. In some embodiments, the entirety of the coating has a thickness of less than 0.1 nm, less than 0.2 nm, less than 0.3 nm, less than 0.4 nm, less than 0.5 nm, less than 0.6 nm, less than 0.7 nm, less than 0.8 nm, less than 0.9 nm, less than 1 nm, less than 2 nm, less than 3 nm, less than 4 nm, less than 5 nm, less than 6 nm, less than 7 nm, less than 8 nm, less than 9 nm, less than 10 nm, less than 15 nm, less than 20 nm, less than 30 nm, less than 40 nm, less than 50 nm, or less than 100 nm. In some embodiments, the entirety of the coating has a thickness of between 10 nm and 50 nm. In some embodiments, the entirety of the coating has a thickness of between 10 nm and 200 nm, between 10 nm and 100 nm, between 10 nm and 50 nm, or between 25 nm and 50 nm. In some embodiments, the entirety of the coating has a thickness of 10-60 nm, 10-50 nm, 10-40 nm or 10-30 nm.

In some embodiments, an individual layer in a multi-layer coating has thickness in the range of 0.1 nm to 100 nm, 0.1 nm to 50 nm, 0.1 nm to 10 nm, 0.1 to 5 nm, 1 nm to 50 nm, 1 nm to 10 nm, or 1 nm to 5 nm. In some embodiments, the aluminum oxide layer has a thickness of more than 0.1 nm, more than 0.2 nm, more than 0.3 nm, more than 0.4 nm, more than 0.5 nm, more than 0.6 nm, more than 0.7 nm, more than 0.8 nm, more than 0.9 nm, more than 1 nm, more than 2 nm, more than 3 nm, more than 4 nm, more than 5 nm, more than 6 nm, more than 7 nm, more than 8 nm, more than 9 nm, more than 10 nm, more than 15 nm, more than 20 nm, more than 30 nm, more than 40 nm, more than 50 nm, or more than 100 nm. In some embodiments, the coating has a thickness of less than 0.1 nm, less than 0.2 nm, less than 0.3 nm, less than 0.4 nm, less than 0.5 nm, less than 0.6 nm, less than 0.7 nm, less than 0.8 nm, less than 0.9 nm, less than 1 nm, less than 2 nm, less than 3 nm, less than 4 nm, less than 5 nm, less than 6 nm, less than 7 nm, less than 8 nm, less than 9 nm, less than 10 nm, less than 15 nm, less than 20 nm, less than 30 nm, less than 40 nm, less than 50 nm, or less than 100 nm. In some embodiments, the coating has a thickness of between 1 nm and 30 nm. In some embodiments, the coating has a thickness of between 1 nm and 20 nm. In some embodiments, the coating has a thickness of 2-5 nm, 5-10 nm, or 10-20 nm. In some embodiments, individual AZO, ASO, ZnOx, AlOx or SiOx layers can have a thickness of 1-10 nm, 5-10 nm, 5-20 nm or 10-20 nm.

As used herein, the terms “approximately” and “about,” as applied to one or more values of interest, refer to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value). For example, when used in the context of an amount of a given compound in a composition, “about” may mean +/−10% of the recited value. For instance, a composition including about 100 ng/ml of a given compound may include 90˜110 ng/ml of the compound.

As used herein, the term “tampering” refers to any manipulation by mechanical, thermal and/or chemical means, which changes the physical properties of the formulation. The tampering can be, for example, crushing, shearing, grinding, chewing, dissolution in a solvent, heating (for example, greater than about 45° C.), or any combination thereof. When the coated antagonist particles are tampered with, the antagonist can be released from the coated antagonist particles.

As used herein, the term “opioid,” “opioid agonist,” or “opioid analgesic” refers to include a drug, hormone, or other chemical or biological substance, natural or synthetic, having a sedative, narcotic, or otherwise similar effect(s) to those containing opium or its natural or synthetic derivatives. The term “opioid agonist” includes one or more opioid agonists, either alone or in combination, and is further meant to include the base of the opioid, mixed or combined agonist-antagonists, partial agonists, pharmaceutically acceptable salts thereof, stereoisomers thereof, ethers thereof, esters thereof, and combinations thereof.

As used herein, the term “antagonist” or “antagonist of a therapeutic agent” refers to any drug or molecule, naturally-occurring or synthetic that negates the effect of the therapeutic agent or produces an unpleasant or punishing stimulus or effect, which will deter or cause avoidance of tampering with the formulation.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

### BRIEF DESCRIPTION OF THE DRAWINGS

FIG. 1 depicts a schematic illustration of an exemplary reactor system.

FIG. 2 depicts a schematic illustration of an exemplary dry blend formulation comprising a coated antagonist particle and a coated opioid particle.

FIG. 3 depicts a schematic illustration of an exemplary supercycle. The exemplary supercycle contains n number of AlOx cycles and m number of ZnOx cycles. The AlOx cycle includes applying a vaporous or gaseous aluminum precursor (e.g., trimethylaluminum or TMA); performing one or more pump-purge cycles of the reactor using an inert gas; applying a vaporous or gaseous oxidant (e.g., H₂O) to the particles in the reactor by pulsing the oxidant into the reactor; and performing one or more pump-purge cycles of the reactor using an inert gas. The ZnOx cycle includes applying a vaporous or gaseous zinc precursor (e.g., diethylzinc or DEZ); performing one or more pump-purge cycles of the reactor using an inert gas; applying a vaporous or gaseous oxidant (e.g., H₂O) to the particles in the reactor by pulsing the oxidant into the reactor; and performing one or more pump-purge cycles of the reactor using an inert gas.

FIG. 4 depicts a schematic illustration of an exemplary cycle in a supercycle. The cycle contains applying a vaporous or gaseous precursor (e.g., TMA or DEZ); performing one or more pump-purge cycles of the reactor using an inert gas; applying a vaporous or gaseous oxidant (e.g., H₂O) to the particles in the reactor by pulsing the oxidant into the reactor; and performing one or more pump-purge cycles of the reactor using an inert gas.

FIG. 5 depicts a schematic illustration of particle coated with multiple layers. In this example, an AlOx layer is applied using conventional cycles, then an AZO layer is applied using supercycles. Finally a ZnOx layer is applied using conventional cycles. The Al/Zn ratio in the AZO layer can be adjusted by varying the numbers of AlOx cycles and ZnOx cycles. The overall coating thickness and the coating wt % can be adjusted by varying the number of conventional cycles and the number of supercycles.

FIG. 6 depicts a schematic illustration of an example of the normal use (administered as prescribed) of a drug formulation comprising coated opioid particles and coated opioid antagonist particles. In this example, the coated opioid particles and coated opioid antagonist particles are mixed together inside of a capsule. When the capsule is ingested normally by a patient, the opioid is released (therapeutic window), while the opioid antagonist is not released or released in a minimal amount (outside the therapeutic window).

FIG. 7 depicts a schematic illustration of the tampering (grinding) of a drug formulation comprising coated opioid particles and coated opioid antagonist particles. In this example, the coated opioid particles and coated opioid antagonist particles are mixed together inside of a capsule. When the capsule is tampered by grinding (e.g., crushed using a mortar and pestle), both the opioid and the opioid antagonist are released, destroying the therapeutic effects.

FIG. 8 depicts a schematic illustration of an example of the tampering (solvent dissolution) of a drug formulation comprising coated opioid particles and coated opioid antagonist particles. The coated opioid particles and coated opioid antagonist particles are mixed together inside of a capsule. When the capsule is tampered by solvent dissolution (e.g., using lemonade or vinegar), both the opioid and the opioid antagonist are released, destroying the therapeutic effects.

### DETAILED DESCRIPTION

This disclosure pertains to methods for preparing coated particles comprising an antagonist-containing core and a coating comprising at least one inorganic oxide. The coating can be applied by vapor phase deposition. The coated particles have a modified drug release profile comparing to the uncoated drug particles.

### Antagonist

As used herein, the term “antagonist” or “antagonist of a therapeutic agent” refers to any drug or molecule, naturally-occurring or synthetic that negates the effect of the therapeutic agent or produces an unpleasant or punishing stimulus or effect, which will deter or cause avoidance of tampering with the formulation.

An antagonist may bind to the same target molecule (e.g., a receptor) of the drug (therapeutic agent), yet does not produce a therapeutic, intracellular, or in vivo response. In this regard, the antagonist of a therapeutic agent binds to the receptor of the therapeutic agent, thereby preventing the therapeutic agent from acting on the receptor. In the case of opioids, an antagonist may prevent the achievement of a “high” in the host.

### Drug

The drug (therapeutic agent or API) can be any medicinal agent used for the treatment of a condition or disease, a pharmaceutically acceptable salt thereof, or an analogue of either of the foregoing. The therapeutic agent may be one that is addictive (physically and/or psychologically) upon repeated use and typically leads to abuse of the therapeutic agent. In this regard, the therapeutic agent can be any opioid agonist as discussed herein.

The drug may be an opioid or an opioid agonist, pharmaceutically acceptable salts thereof, stereoisomers thereof, ethers thereof, esters thereof, and combinations thereof. The opioid agonist may be alfentanil, allylprodine, alphaprodine, anileridine, benzylmorphine, bezitramide, buprenorphine, butorphanol, clonitazene, codeine, cyclazocine, desomorphine, dextromoramide, dezocine, diampromide, dihydrocodeine, dihydroetorphine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazene, etorphine, fentanyl, heroin, hydrocodone, hydromorphone, hydroxypethidine, isomethadone, ketobemidone, levallorphan, levorphanol, levophenacylmorphan, lofentanil, meperidine, meptazinol, metazocine, methadone, metopon, morphine, myrophine, nalbuphine, narceine, nicomorphine, norlevorphanol, normethadone, nalorphine, normorphine, norpipanone, opium, oxycodone, oxymorphone, papavereturn, pentazocine, phenadoxone, phenazocine, phenomorphan, phenoperidine, piminodine, piritramide, propheptazine, promedol, properidine, propiram, propoxyphene, sufentanil, tramadol, tilidine, derivatives or complexes thereof, pharmaceutically acceptable salts thereof, or combinations thereof. The opioid agonist may be selected from the group consisting of hydrocodone, hydromorphone, oxycodone, dihydrocodeine, codeine, dihydromorphine, morphine, buprenorphine, derivatives or complexes thereof, pharmaceutically acceptable salts thereof, and combinations thereof. The opioid agonist may be morphine, hydromorphone, oxycodone or hydrocodone. The opioid agonist may comprise oxycodone or hydrocodone (e.g., in an amount of about 15 to about 45 mg in the formulation), and the opioid antagonist may comprises naltrexone (e.g., in an amount of about 0.5 to about 5 mg in the formulation).

Hydrocodone is a semisynthetic narcotic analgesic and antitussive with multiple nervous system and gastrointestinal actions. Chemically, hydrocodone is 4,5-epoxy-3-methoxy-17-methylmorphinan-6-one, and is also known as dihydrocodeinone. Like other opioids, hydrocodone can be habit-forming and can produce drug dependence of the morphine type. Like other opium derivatives, excess doses of hydrocodone will depress respiration.

Oxycodone, chemically known as 4,5-epoxy-14-hydroxy-3-methoxy-17-methylmorphinan-6-one, is an opioid agonist whose principal therapeutic action is analgesia. Other therapeutic effects of oxycodone include anxiolysis, euphoria and feelings of relaxation. The precise mechanism of its analgesic action is not known, but specific CNS opioid receptors for endogenous compounds with opioid-like activity have been identified throughout the brain and spinal cord and play a role in the analgesic effects of this drug. Oxycodone is commercially available in the United States, e.g., as Oxycotin® from Purdue Pharma L.P. (Stamford, Conn.), as controlled-release tablets for oral administration containing 10 mg, 20 mg, 40 mg or 80 mg oxycodone hydrochloride, and as OxyIR™, also from Purdue Pharma L.P., as immediate-release capsules containing 5 mg oxycodone hydrochloride.

Exemplary NMDA receptor medicaments include morphinans, such as dextromethorphan or dextrophan, ketamine, d-methadone, and pharmaceutically acceptable salts thereof, and encompass drugs that block a major intracellular consequence of NMDA-receptor activation, e.g., a ganglioside, such as (6-aminothexyl)-5-chloro-1-naphthalenesulfonamide. These drugs are stated to inhibit the development of tolerance to and/or dependence on addictive drugs, e.g., narcotic analgesics, such as morphine, codeine; etc., in U.S. Pat. Nos. 5,321,012 and 5,556,838 (both to Mayer et al.), both of which are incorporated herein by reference, and to treat chronic pain in U.S. Pat. No. 5,502,058 (Mayer et al.), incorporated herein by reference. The NMDA agonist can be included alone or in combination with a local anesthetic, such as lidocaine, as described in these patents by Mayer et al.

### Uncoated Particles

The uncoated particles may be antagonist particles (particles of one or more antagonists). The uncoated particles may be drug particles (particles of one or more drugs). The uncoated particles may comprise (1) antagonist particles (particles of one or more antagonists) and (2) drug particles (particles of one or more drugs). The uncoated particles may further contain one or more pharmaceutically acceptable excipients.

The uncoated particles may contain at least 10%, 20%, 30%, 40%, or 50% wt/wt antagonists. The uncoated particles may contain less than 10%, 20%, 30%, 40%, or 50% wt/wt antagonists. The uncoated particles may contain at least 60%, 70%, 80%, 90%, 99% or 100% wt/wt antagonists.

The uncoated particles may contain at least 10%, 20%, 30%, 40%, or 50% wt/wt drugs. The uncoated particles may contain less than 10%, 20%, 30%, 40%, or 50% wt/wt drugs. The uncoated particles may contain at least 60%, 70%, 80%, 90%, 99% or 100% wt/wt drugs.

The uncoated particles may have a D10 of less than 0.1 μm, less than 0.2 μm, less than 0.5 μm, less than 1 μm, less than 2 μm, less than 5 μm, less than 10 μm, less than 20 μm, or less than 50 μm, on a volume average basis. The uncoated particles may have a D10of more than 0.1 μm, more than 0.2 μm, more than 0.5 μm, more than 1 μm, more than 2 μm, more than 5 μm, more than 10 μm, more than 20 μm, or more than 50 μm, on a volume average basis. The uncoated particles may have a D10 of 0.1 μm to 200 μm, 0.1 μm to 1 μm, 0.1 μm to 10 μm, or 0.1 μm to 50 μm on a volume average basis. The uncoated particles may have a D10 of about 2 μm on a volume average basis.

The uncoated particles may have a D50 of less than 0.1 μm, less than 0.2 μm, less than 0.5 μm, less than 1 μm, less than 2 μm, less than 5 μm, less than 10 μm, less than 20 μm, or less than 50 μm, on a volume average basis. The uncoated particles may have a D50of more than 0.1 μm, more than 0.2 μm, more than 0.5 μm, more than 1 μm, more than 2 μm, more than 5 μm, more than 10 μm, more than 20 μm, or more than 50 μm, on a volume average basis. The uncoated particles may have a D50 of 0.1 μm to 200 μm, 0.1 μm to 1 μm, 0.1 μm to 10 μm, or 0.1 μm to 50 μm on a volume average basis. The uncoated particles may have a D50 of about 4.5 μm on a volume average basis.

The uncoated particles may have a D90 of less than 0.1 μm, less than 0.2 μm, less than 0.5 μm, less than 1 μm, less than 2 μm, less than 5 μm, less than 10 μm, less than 20 μm, or less than 50 μm, on a volume average basis. The uncoated particles may have a D90of more than 0.1 μm, more than 0.2 μm, more than 0.5 μm, more than 1 μm, more than 2 μm, more than 5 μm, more than 10 μm, more than 20 μm, or more than 50 μm, on a volume average basis. The uncoated particles may have a D90 of 200 μm to 2000 μm on a volume average basis. The uncoated particles may have a D50 of 0.1 μm to 200 μm, 0.1 μm to 1 μm, 0.1 μm to 10 μm, or 0.1 μm to 50 μm on a volume average basis. The uncoated particles may have a D90 of about 9.2 μm on a volume average basis.

### Vapor Phase Deposition

In order to control the release of antagonists and/or drugs, coatings are applied to the uncoated particles by vapor phase deposition using a precursor molecule (e.g., an inorganic oxide precursor) and an oxidant (e.g., ozone or water vapor). Vapor phase deposition of inorganic oxides (e.g., metal oxides or metalloid oxides) is sometimes referred to as atomic layer deposition (ALD). However, depending on a number of factors, including the surface being coated, each cycle of the deposition reaction does not necessarily deposit one atomic layer on the entire surface.

### Reactor System

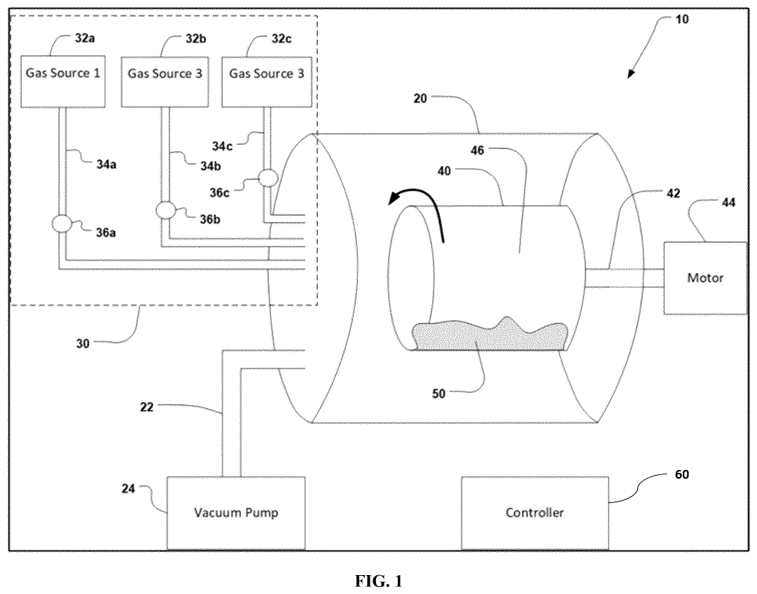

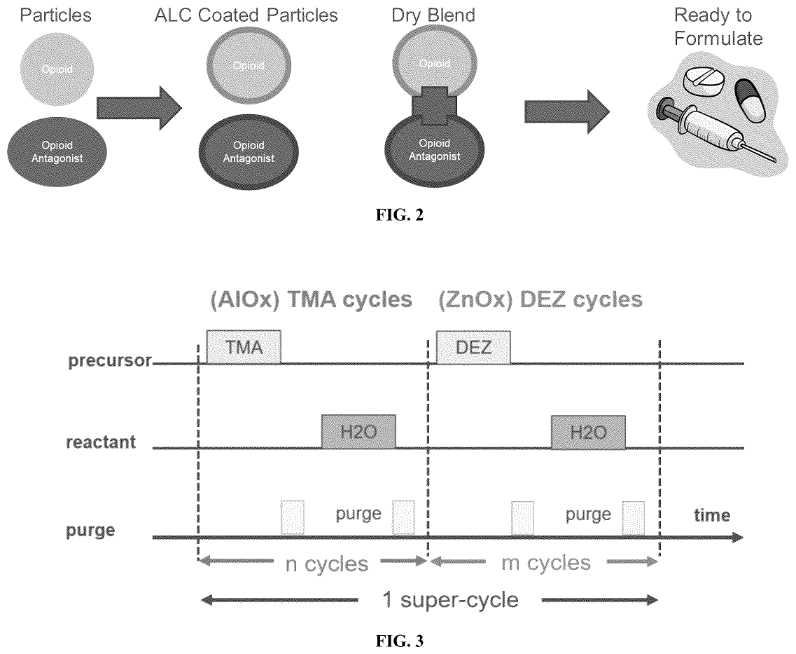

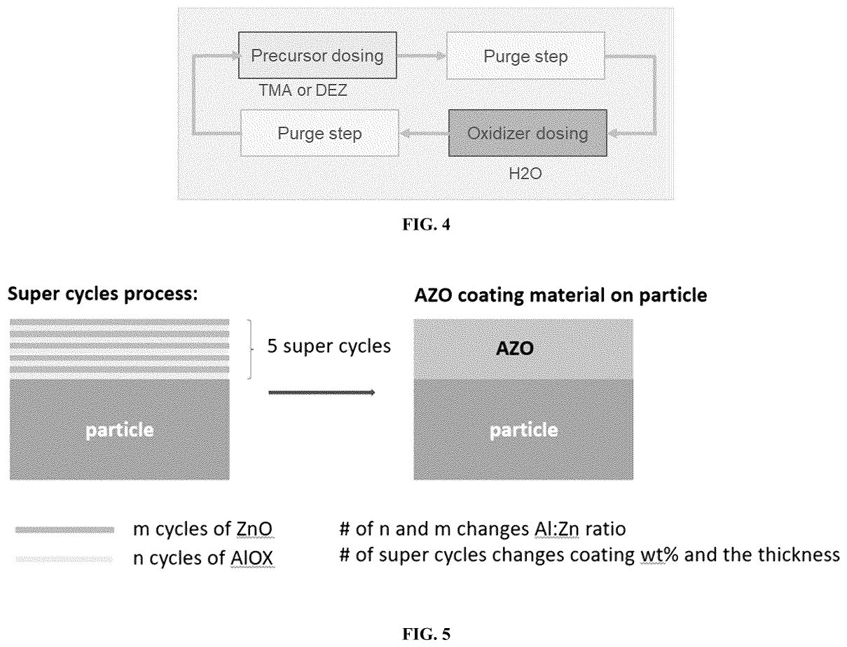

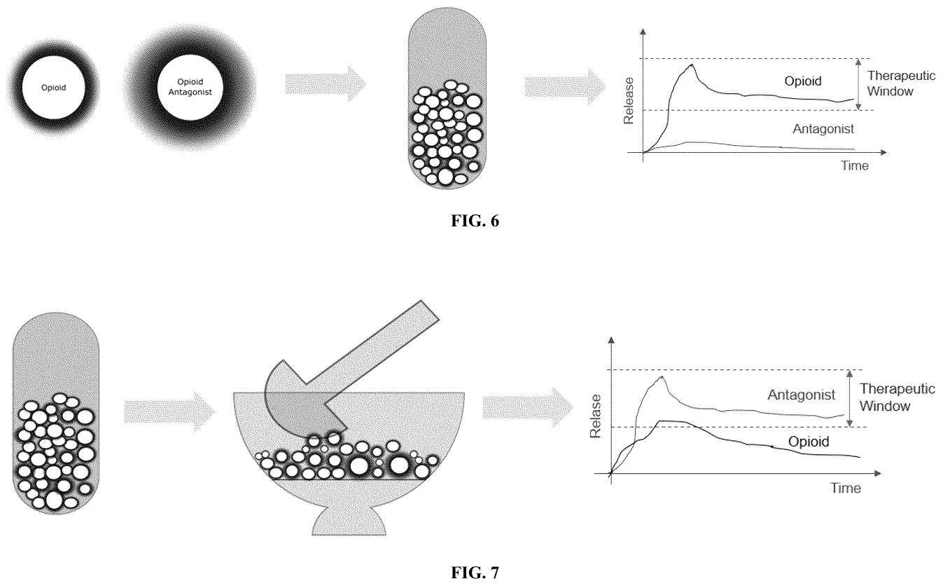

The term “reactor system” in its broadest sense includes all systems that could be used to perform vapor phase deposition or atomic layer deposition. An exemplary reactor system is illustrated in FIG. 1 and further described below.

The reactor system 10 can perform vapor phase deposition or atomic layer deposition. The reactor system 10 permits the process to be performed at higher (above 50° C., e.g., 50-100° C. or higher) or lower process temperature, e.g., below 50° C., e.g., at or below 25° C. For example, the reactor system 10 can form thin-film inorganic oxides on the particles primarily at temperatures of 40-80° C., e.g., 40° C. or 80° C. In general, the particles can remain or be maintained at such temperatures. This can be achieved by having the reactants and/or the interior surfaces of the reactor chamber (e.g., the chamber 20 and drum 40 discussed below) remain or be maintained at such temperatures.

Again, illustrating a vapor phase deposition or atomic layer deposition process, the reactor system 10 includes a stationary vacuum chamber 20 which is coupled to a vacuum pump 24 by vacuum tubing 22. The vacuum pump 24 can be an industrial vacuum pump sufficient to establish pressures less than 1 Torr, e.g., 1 to 100 mTorr, e.g., 50 mTorr. The vacuum pump 24 permits the chamber 20 to be maintained at a desired pressure and permits removal of reaction byproducts and unreacted process gases.

In operation, the reactor 10 performs the vapor phase deposition or atomic layer deposition process by introducing a gaseous oxidant and aluminum (or zinc) precursor into the chamber 20. The gaseous oxidant and aluminum (or zinc) precursor are introduced alternatively into the reactor. In addition, the reaction can be performed at low temperature conditions, such as below 80° C., e.g., below 50° C., below 30° C., or below 25° C. The operating temperature may be 50° C. The operating temperature may be above 5° C., above 10° C., above 15° C., above 20° C., above 25° C., above 30° C., above 35° C., above 40° C., above 45° C., above 50° C., above 56° C., above 60° C., above 65° C., above 70° C., above 75° C., or above 80° C. The operating temperature may be below 20° C., below 25° C., below 30° C., below 35° C., below 40° C., below 45° C., below 50° C., below 56° C., below 60° C., below 65° C., below 70° C., below 75° C., or below 80° C.

The chamber 20 is also coupled to a chemical delivery system 30. The chemical delivery system 30 includes three or more gas sources 32a, 32b, 32c coupled by respective delivery lines 34a, 34b, 34c and controllable valves 36a, 36b, 36c to the vacuum chamber 20. The chemical delivery system 30 can include a combination of restrictors, gas flow controllers, pressure transducers, and ultrasonic flow meters to provide controllable flow rate of the various gasses into the chamber 20. The chemical delivery system 30 can also include one or more temperature control components, e.g., a heat exchanger, resistive heater, heat lamp, etc., to heat or cool the various gasses before they flow into the chamber 20. Although FIG. 1 illustrates separate gas lines extending in parallel to the chamber for each gas source, two or more of the gas lines could be joined, e.g., by one or more three-way valves, before the combined line reaches the chamber 20.

One of the gas sources can provide an oxidant. In particular, a gas source can provide a vaporous or gaseous oxidant. For example, the oxidant can be ozone. As another example, the oxidant can be water vapor.

One of the gas sources can be an aluminum (or zinc) precursor. In particular, a gas source can provide a vaporous or gaseous aluminum (or zinc) precursor. For example, the aluminum precursor can be TMA.

One of the gas sources can provide a purge gas. In particular, the third gas source can provide a gas that is chemically inert to the oxidant and aluminum (or zinc) precursor, the coating, and the particles being processed. For example, the purge gas can be N₂, or a noble gas, such as argon.

A rotatable coating drum 40 is held inside the chamber 20. The drum 40 can be connected by a drive shaft 42 that extends through a sealed port in a side wall of the chamber 20 to a motor 44. The motor 44 can rotate the drum at speeds of 1 to 100 rpm. Alternatively, the drum can be directly connected to a vacuum source through a rotary union.

The particles to be coated, shown as a particle bed 50, are placed in an interior volume 46 of the drum 40. The drum 40 and chamber 20 can include sealable ports (not illustrated) to permit the particles to be placed into and removed from the drum 40.

The body of the drum 40 is provided by one or more of a porous material, a solid metal, and a perforated metal. The pores through the cylindrical side walls of the drum 40 can have a dimension of 1-10 μm.

In operation, one of the gasses flows into chamber 20 from the chemical delivery system 30 as the drum 40 rotates. A combination of pores (1-100 um), holes (0.1-10 mm), or large openings in the coating drum 40 serve to confine the particles in the coating drum 40 while allowing rapid delivery of precursor chemistry and the pumping of byproducts or unreacted species. Due to the pores in the drum 40, the gas can flow between the exterior of the drum 40, i.e., the reactor chamber 20, and the interior of the drum 40. In addition, rotation of the drum 40 agitates the particles to expose new surfaces of the powder bed, ensuring a large surface area of the particles remains exposed to the process gas. This permits fast, uniform interaction of the particle surface with the process gas.

In some implementations, one or more temperature control components are integrated into the drum 40 to permit control of the temperature of the drum 40. For example, a resistive heater, a thermoelectric cooler, or other component can be in or on the side walls of the drum 40.

The reactor system 10 also includes a controller 60 coupled to the various controllable components, e.g., vacuum pump 24, gas distribution system 30, motor 44, a temperature control system, etc., to control operation of the reactor system 10. The controller 60 can also be coupled to various sensors, e.g., pressure sensors, flow meters, etc., to provide closed loop control of the pressure of the gasses in the chamber 20.

In general, the controller 60 can operate the reactor system 10 in accord with a “recipe.” The recipe specifies an operating value for each controllable element as a function of time. For example, the recipe can specify the times during which the vacuum pump 24 is to operate, the times of and flow rate for each gas source 32a, 32b, 32c, the rotation rate of the motor 44, etc. The controller 60 can receive the recipe as computer-readable data (e.g., that is stored on a non-transitory computer readable medium).

The controller 60 and other computing device parts of systems described herein can be implemented in digital electronic circuitry, or in computer software, firmware, or hardware. For example, the controller can include a processor to execute a computer program as stored in a computer program product, e.g., in a non-transitory machine-readable storage medium. Such a computer program (also known as a program, software, software application, or code) can be written in any form of programming language, including compiled or interpreted languages, and it can be deployed in any form, including as a standalone program or as a module, component, subroutine, or other unit suitable for use in a computing environment. In some implementations, the controller 60 is a general-purpose programmable computer. In some implementations, the controller can be implemented using special purpose logic circuitry, e.g., an FPGA (field programmable gate array) or an ASIC (application specific integrated circuit).

### Operation

Initially, uncoated particles (e.g., antagonist particles) are loaded into the drum 40 in the reactor system 10. Once any access ports are sealed, the controller 60 operates the reactor system 10 according to the recipe in order to form the thin-film inorganic oxides on the particles.

In one example, the uncoated particles (e.g., antagonist particles) are coated with an inorganic oxide coating. The uncoated particles can be coated with two reactant gases. The uncoated particles can be coated with a first reactant gas in the form of an inorganic precursor gas and a second reactant gas in the form of an oxidant gas. The inorganic oxide may be aluminum oxide, zinc oxide, titanium oxide, or silicon oxide. The inorganic oxide may be aluminum oxide. The inorganic precursor may be trimethylaluminium (TMA). The inorganic oxide may be zinc oxide. The inorganic precursor may be diethylzinc. The inorganic oxide may be titanium oxide. The inorganic precursor may be titanium tetrachloride (TiCl4). The inorganic oxide may be silicon oxide. The inorganic precursor may be 1,2-Bis (diisopropylamino) disilane (BDIPADS) or silicon tetrachloride. The oxidant may be water or ozone.

In particular, the two reactant gases are alternately supplied to the chamber 20, with each step of supplying a reactant gas followed by a purge cycle in which the inert gas is supplied to the chamber 20 to force out the reactant gas and by-products used in the prior step. Moreover, one or more of the gases (e.g., the reactant gases and/or the inert gas) can be supplied in pulses in which the chamber 20 is filled with the gas to a specified pressure, a delay time is permitted to pass, and the chamber is evacuated by the vacuum pump 24 before the next pulse commences.

In particular, the controller 60 can operate the reactor system 10 as follows.

In a first reactant cycle (called a half-cycle), while the motor 44 rotates the drum 40 to agitate the particles 50:
i) The gas distribution system 30 is operated to flow the first reactant gas, e.g., TMA, from the source 32a into the chamber 20 until a first specified pressure is achieved. The specified pressure can be 0.1 Torr to half of the saturation pressure of the reactant gas.
ii) Flow of the first reactant is halted, and a specified delay time is permitted to pass, e.g., as measured by a timer in the controller. This permits the first reactant to flow through the particle bed in the drum 40 and react with the surface of the particles 50 inside the drum 40.
iii) The vacuum pump 50 evacuates the chamber 20, e.g., down to pressures below 1 Torr, e.g., to 1 to 100 mTorr, e.g., 50 mTorr. These steps (i)-(iii) can be repeated a number of times set by the recipe, e.g., two to ten times, e.g., six times.

Next, in a first purge cycle, while the motor 44 rotates the drum to agitate the particles 50:
iv) The gas distribution system 30 is operated to flow the inert gas, e.g., N₂, from the source 32c into the chamber 20 until a second specified pressure is achieved. The second specified pressure can be 1 to 100 Torr.
v) Flow of the inert gas is halted, and a specified delay time is permitted to pass, e.g., as measured by the timer in the controller. This permits the inert gas to flow through the pores in the drum 40 and diffuse through the particles 50 to displace the reactant gas and any vaporous by-products.
vi) The vacuum pump 50 evacuates the chamber 20, e.g., down to pressures below 1 Torr, e.g., to 1 to 500 mTorr, e.g., 50 mTorr. These steps (iv)-(vi) can be repeated a number of times set by the recipe, e.g., six to twenty times, e.g., sixteen times. Taken together steps (iv)-(vi) are called a pump-purge cycle.

In a second reactant half-cycle, while the motor 44 rotates the drum 40 to agitate the particles 50:

vii) The gas distribution system 30 is operated to flow the second reactant gas, e.g., H2O, from the source 32b into the chamber 20 until a third specified pressure is achieved. The third pressure can be 0.1 Torr to half of the saturation pressure of the reactant gas.
viii) Flow of the second reactant is halted, and a specified delay time is permitted to pass, e.g., as measured by the timer in the controller. This permits the second reactant to flow through the pores in the drum 40 and react with the surface of the particles 50 inside the drum 40.
ix) The vacuum pump 50 evacuates the chamber 20, e.g., down to pressures below 1 Torr, e.g., to 1 to 500 mTorr, e.g., 50 mTorr.

These steps (vii)-(ix) can be repeated a number of times set by the recipe, e.g., two to ten times, e.g., six times.

Next, a second purge cycle is performed. This second purge cycle can be identical to the first purge cycle, or can have a different number of repetitions of the steps (iv)-(vi) and/or different delay time and/or different pressure. The cycle of the first reactant half-cycle, first purge cycle, second reactant half cycle and second purge cycle can be repeated a number of times set by the recipe, e.g., one to ten times.

The uncoated particle may be coated with one or more inorganic oxide coatings, for example, the uncoated particle may be coated with a combination of aluminum oxide, titanium oxide, zinc oxide and/or silicon oxide coatings. The uncoated particles may be first coated with an aluminum oxide layer and then coated with a silicon oxide layer. The uncoated particle may be first coated with an aluminum oxide layer, then coated with a silicon oxide layer, and then coated with an aluminum oxide layer.

Exemplary methods for applying aluminum oxide, titanium oxide, zinc oxide, silicon oxide coatings, multiple coating layers and supercycle coating methods are provided below.

### Methods for Aluminum Oxide Coating

In one aspect, the disclosure provides methods for preparing abuse-deterrent compositions comprising an antagonist particle encapsulated by aluminum oxide coating. The aluminum oxide coating can be performed on a rotary powder coating chamber. The operating temperature can be 35-50° C. or about 50° C. For each aluminum oxide coating process, one precursor may be introduced to reach a pressure of 0.3-2 torr for a hold time of 60 seconds, before nitrogen gas is used to purge the excess reactants and side products. The second precursor may then be introduced to reach a pressure of 2-8 torr for a hold time of 60 seconds, before nitrogen gas is used to purge the excess reactants and side products. This completes one coating cycle. Desired cycle numbers can be decided and aluminum oxide coating can be coated in repeated cycles to obtain a desired thickness.

The aluminum oxide coating can be applied using vapor phase deposition as described herein. The aluminum precursors can be trimethylaluminum (TMA). The oxidant can be water.

A first exemplary aluminum oxide coating method includes the sequential steps of: (a) loading the uncoated particles into a reactor, (b) applying a vaporous or gaseous aluminum precursor (e.g., TMA) to the substrate in the reactor, (c) performing one or more pump-purge cycles of the reactor using inert gas, (d) applying a vaporous or gaseous oxidant (e.g., water) to the substrate in the reactor, and (e) performing one or more pump-purge cycles of the reactor using inert gas. The sequential steps (b)-(e) may be repeated one or more times to increase the total thickness of the aluminum oxide that enclose the solid core of the coated particles. The reactor pressure may be allowed to stabilize following step (a), step (b), and/or step (d). The reactor contents may be agitated prior to and/or during step (b), step (c), and/or step (e). A subset of vapor or gaseous content may be pumped out prior to step (c) and/or step (e).

A second exemplary aluminum oxide coating method includes (e.g., consists of) the sequential steps of (a) loading the uncoated particles into a reactor, (b) reducing the reactor pressure to less than 50 m Torr, (c) agitating the reactor contents until the reactor contents have a desired moisture content, (d) pressurizing the reactor to at least 0.3 Torr by adding a vaporous or gaseous aluminum precursor (e.g., TMA), (e) allowing the reactor pressure to stabilize, (f) agitating the reactor contents, (g) pumping out a subset of vapor or gaseous content and determining when to stop pumping based on analysis of content in reactor, (h) performing a sequence of pump-purge cycles of the reactor using insert gas, (i) pressuring the reactor to 2 Torr by adding a vaporous or gaseous oxidant (e.g., water), (j) allowing the reactor pressure to stabilize, (k) agitating the reactor contents, (l) pumping out a subset of vapor or gaseous content and determining when to stop pumping based on analysis of content in reactor and (m) performing a sequence of pump-purge cycles of the reactor using insert gas. The sequential steps (b)-(m) may be repeated one or more times to increase the total thickness of the one or more aluminum oxide materials that enclose the solid core of the coated particles.

### Methods for Titanium Oxide Coating

In one aspect, the disclosure provides methods for preparing abuse-deterrent compositions comprising an antagonist particle encapsulated by titanium oxide coating.

The titanium oxide coating can be performed on a rotary powder coating chamber. The operating temperature can be 35-50° C. or about 50° C. For each titanium oxide coating process, one precursor may be introduced to reach a pressure of 0.3-2 torr for a hold time of 60 seconds, before nitrogen gas is used to purge the excess reactants and side products. The second precursor may then be introduced to reach a pressure of 2-8 torr for a hold time of 60 seconds, before nitrogen gas is used to purge the excess reactants and side products. This completes one coating cycle. Desired cycle numbers can be decided and titanium oxide coating can be coated in repeated cycles to obtain a desired thickness.

The titanium oxide coating can be applied using vapor phase deposition as described herein. The titanium precursor can be titanium tetrachloride (TiCl₄), tetrakis (dimethylamino) titanium (TDMAT), tetrakis (diethylamino) titanium (TDEAT), or tetrakis (ethylmethylamino) titanium (TEMAT). The oxidant can be water or ozone. The titanium precursor may be titanium tetrachloride and the oxidant may be water.

A first exemplary titanium oxide coating method includes the sequential steps of: (a) loading the uncoated particles into a reactor, (b) applying a vaporous or gaseous titanium precursor (e.g., TiCl₄) to the substrate in the reactor, (c) performing one or more pump-purge cycles of the reactor using inert gas, (d) applying a vaporous or gaseous oxidant (e.g., water) to the substrate in the reactor, and (e) performing one or more pump-purge cycles of the reactor using inert gas. The sequential steps (b)-(e) may be repeated one or more times to increase the total thickness of the titanium oxide that enclose the solid core of the coated particles. The reactor pressure may be allowed to stabilize following step (a), step (b), and/or step (d). The reactor contents may be agitated prior to and/or during step (b), step (c), and/or step (e). A subset of vapor or gaseous content may be pumped out prior to step (c) and/or step (e).

A second exemplary titanium oxide coating method includes (e.g., consists of) the sequential steps of (a) loading the uncoated particles into a reactor, (b) reducing the reactor pressure to less than 50 m Torr, (c) agitating the reactor contents until the reactor contents have a desired moisture content, (d) pressurizing the reactor to at least 0.3 Torr by adding a vaporous or gaseous titanium precursor (e.g., TiCl₄), (e) allowing the reactor pressure to stabilize, (f) agitating the reactor contents, (g) pumping out a subset of vapor or gaseous content and determining when to stop pumping based on analysis of content in reactor, (h) performing a sequence of pump-purge cycles of the reactor using insert gas, (i) pressuring the reactor to 2 Torr by adding a vaporous or gaseous oxidant (e.g., water), (j) allowing the reactor pressure to stabilize, (k) agitating the reactor contents, (l) pumping out a subset of vapor or gaseous content and determining when to stop pumping based on analysis of content in reactor and (m) performing a sequence of pump-purge cycles of the reactor using insert gas. The sequential steps (b)-(m) may be repeated one or more times to increase the total thickness of the one or more titanium oxide materials that enclose the solid core of the coated particles.

Methods for Zinc Oxide Coating In one aspect, the disclosure provides methods for preparing abuse-deterrent compositions comprising an antagonist particle encapsulated by zinc oxide coating.

The first exemplary method includes the sequential steps of: (a) loading the uncoated particles into a reactor, (b) applying a vaporous or gaseous zinc precursor to the substrate in the reactor, (c) performing one or more pump-purge cycles of the reactor using inert gas, (d) applying a vaporous or gaseous oxidant (e.g., water) to the substrate in the reactor, and (e) performing one or more pump-purge cycles of the reactor using inert gas. The sequential steps (b)-(e) can be repeated one or more times to increase the total thickness of the zinc oxide that enclose the solid core of the coated particles. The reactor pressure may be allowed to stabilize following step (a), step (b), and/or step (d). The reactor contents may be agitated prior to and/or during step (b), step (c), and/or step (e). A subset of vapor or gaseous content may be pumped out prior to step (c) and/or step (e).

The second exemplary method includes (e.g., consists of) the sequential steps of (a) loading the uncoated particles into a reactor, (b) reducing the reactor pressure to less than 50 m Torr, (c) agitating the reactor contents until the reactor contents have a desired moisture content, (d) pressurizing the reactor to at least 0.3 Torr by adding a vaporous or gaseous zinc precursor, (e) allowing the reactor pressure to stabilize, (f) agitating the reactor contents, (g) pumping out a subset of vapor or gaseous content and determining when to stop pumping based on analysis of content in reactor, (h) performing a sequence of pump-purge cycles of the reactor using insert gas, (i) pressuring the reactor to 2 Torr by adding a vaporous or gaseous oxidant (e.g., water), (j) allowing the reactor pressure to stabilize, (k) agitating the reactor contents, (l) pumping out a subset of vapor or gaseous content and determining when to stop pumping based on analysis of content in reactor and (m) performing a sequence of pump-purge cycles of the reactor using insert gas. The sequential steps (b)-(m) can be repeated one or more times to increase the total thickness of the one or more zinc oxide materials that enclose the solid core of the coated particles.

The atomic layer coating process may comprise: (b1) loading the uncoated particles into a reactor; (b2) applying a vaporous or gaseous zinc precursor to the particles in the reactor; (b3) performing one or more pump-purge cycles of the reactor using inert gas; (b4) applying a vaporous or gaseous oxidant (e.g., water) to the particles in the reactor; and (b5) performing one or more pump-purge cycles of the reactor using inert gas. Steps (b2)-(b5) can be performed two or more times to increase the total thickness of the zinc oxide layer before step (c) is performed.

The reactor pressure may be allowed to stabilize following step (b1), step (b2), and/or step (b4). The reactor contents may be agitated prior to and/or during step (b1), step (b3), and/or step (b5). A subset of vapor or gaseous content may be pumped out prior to step (b3) and/or step (b5). Step (b) may take place at a temperature between 45° C. and 55° C.

### Methods for Silicon Oxide Coating

In one aspect, the disclosure provides methods for preparing abuse-deterrent compositions comprising an antagonist particle encapsulated by silicon oxide coating.

The silicon oxide coating can be applied using vapor phase deposition as described herein. The silicon precursors can be silicon tetrachloride or 1,2-Bis (diisopropylamino) disilane (BDIPADS). The oxidant can be water or ozone.

A first exemplary silicon oxide coating method includes the sequential steps of: (a) loading the uncoated particles into a reactor, (b) applying a vaporous or gaseous silicon precursor (e.g., silicon tetrachloride, BDIPADS) to the substrate in the reactor, (c) performing one or more pump-purge cycles of the reactor using inert gas, (d) applying a vaporous or gaseous oxidant (e.g., ozone) to the substrate in the reactor, and (e) performing one or more pump-purge cycles of the reactor using inert gas. The sequential steps (b)-(e) may be repeated one or more times to increase the total thickness of the silicon oxide that enclose the solid core of the coated particles. The reactor pressure may be allowed to stabilize following step (a), step (b), and/or step (d). The reactor contents may be agitated prior to and/or during step (b), step (c), and/or step (e). A subset of vapor or gaseous content may be pumped out prior to step (c) and/or step (e).

A second exemplary silicon oxide coating method includes (e.g., consists of) the sequential steps of (a) loading the uncoated particles into a reactor, (b) reducing the reactor pressure to less than 50 m Torr, (c) agitating the reactor contents until the reactor contents have a desired moisture content, (d) pressurizing the reactor to at least 0.3 Torr by adding a vaporous or gaseous silicon precursor (e.g., silicon tetrachloride, BDIPADS), (e) allowing the reactor pressure to stabilize, (f) agitating the reactor contents, (g) pumping out a subset of vapor or gaseous content and determining when to stop pumping based on analysis of content in reactor, (h) performing a sequence of pump-purge cycles of the reactor using insert gas, (i) pressuring the reactor to 2 Torr by adding a vaporous or gaseous oxidant (e.g., ozone), (j) allowing the reactor pressure to stabilize, (k) agitating the reactor contents, (l) pumping out a subset of vapor or gaseous content and determining when to stop pumping based on analysis of content in reactor and (m) performing a sequence of pump-purge cycles of the reactor using insert gas. The sequential steps (b)-(m) may be repeated one or more times to increase the total thickness of the one or more silicon oxide materials that enclose the solid core of the coated particles.

Methods for Preparing a Coated Particle with Multiple Coating Layers

In some cases, the coating is applied using two or more different processes, for example, a silicon oxide coating process and an aluminum oxide coating process. The two or more different processes can be combined, e.g., to provide a distinct, inner aluminum oxide coating layer adjacent to the protein particle and a distinct, outer silicon oxide coating layer that encloses the particle, including the inner layer. In some cases, another coating layer can be applied surrounding the outer coating layer. Thus, there can be a distinct inner layer of a first metal or metalloid oxide, a distinct intermediate layer of a second metal or metalloid oxide and a third outer layer of the first (or a third) metalloid oxide. In general, the formation of such distinct coating layers entails at least 10 cycles of vapor phase deposition for each distinct coating layer, generating a coating layer that is about 2.5-100 nanometers thick.

### Methods for Preparing a Coated Particle by Supercycles

In some cases, the coating is applied using two or more different processes, for example, a silicon oxide coating process and an aluminum oxide coating process. The two or more different processes can be combined to provide a coating that include a ternary compound layer (e.g., an aluminum-zinc-oxide layer or AZO layer) that more closely resembles combinations of two different metal or metalloid oxides, as opposed to distinct multi-layer coatings having multiple, distinct metal or metalloid coating layers (described above). A so-called supercycle process is used to produce ternary compound layers. In the supercycle process, only a very limited number of cycles (e.g., less than 10, 1-10, 2-10, 3-10, 1-5, 2-8, 2-8 cycles) are carried out with a first precursor before switching to a second precursor. Each supercycle includes a number of cycles with a first precursor and a number of cycles with a second precursor. The relatively frequent alteration in precursor during a supercycle process produces a coating that does not have distinct layers. Thus, a coating layer prepared by carrying out a small number (e.g., 1-10) of cycles using an aluminum oxide precursor alternating with a small number of cycles using a zinc oxide precursor can be referred to as an AZO layer. Importantly, the composition of the ternary compound layer can adjusted by having different numbers cycles with each precursor, e.g., alternating 3 cycles with an aluminum oxide precursor and 2 cycles with a zinc oxide precursor or alternating 2 cycles with an aluminum oxide precursor and 5 cycles with a zinc oxide precursor. when 2 or more supercycle steps are carried out, the number of cycles with each precursor do not need to remain the same. Thus, there can be a first supercycle having 3 cycles with an aluminum oxide precursor and 2 cycles with a zinc oxide precursor followed by a second supercycle with 2 cycles with an aluminum oxide precursor and 5 cycles with a zinc oxide precursor.

Of course, a supercycle coating process can be combined with a more conventional coating process. Thus, a particle can have an inner layer produced using a supercycle process and an outer layer that is a distinct layer (i.e., not a ternary compound layer). The two types of coating layers can be applied in reverse order to produce a particle having an inner layer that is a distinct layer (i.e., not a ternary compound layer) and outer layer that is a ternary compound layer produced using a supercycle process.

In one example, the disclosure provides methods to prepare a coated particle that has a core and at least one coating layer applied using supercycles, each supercycle having a first number of first cycles and a second number of second cycles. The methods include the sequential steps of: (a) providing uncoated particles; (b) performing a first number of first cycles; using first inorganic oxide precursor and (c) performing a second number of second cycles using a second inorganic oxide precursor, wherein the first and second inorganic oxide precursors are for forming different inorganic oxides (e.g., the first precursor can be aluminum oxide precursor and the second precursor can be zinc oxide precursor). The vaporous or gaseous oxidant used in the first and second cycles can be the same or different.

The step of performing a first number of first cycles (step (b)) comprises: (b1) applying a vaporous or gaseous first inorganic oxide precursor to the particles in the reactor by pulsing the vaporous or gaseous first inorganic oxide precursor into the reactor; (b2) performing one or more pump-purge cycles of the reactor using an inert gas; (b3) applying a vaporous or gaseous oxidant to the particles in the reactor by pulsing the oxidant into the reactor; and (b4) performing one or more pump-purge cycles of the reactor using an inert gas.

The step of performing a second number of second cycles (step (c)) comprises: (c1) applying a vaporous or gaseous second inorganic oxide precursor to the particles in the reactor by pulsing the vaporous or gaseous second inorganic oxide precursor into the reactor; (c2) performing one or more pump-purge cycles of the reactor using an inert gas; (c3) applying a vaporous or gaseous oxidant to the particles in the reactor by pulsing the oxidant into the reactor; and (c4) performing one or more pump-purge cycles of the reactor using an inert gas.

The steps (b)-(c) constitutes a supercycle. Steps (b)-(c) can be performed two or more times to increase the total thickness of the coating. The particles can be agitated prior to and/or during step (a). The reactor pressure can be allowed to stabilize following step (b1), step (b2), step (b3) and/or step (b4). The reactor pressure can be allowed to stabilize following step (c1), step (c2), step (c3) and/or step (c4).

For example, aluminum oxide and zinc oxide precursors can be applied using supercycles to create an AZO layer (see, e.g., FIG. 3). The aluminum precursor can be trimethylaluminum (TMA). The zinc precursor can be diethylzinc (DEZ) or zinc tetrachloride.

As another example, aluminum oxide and silicon oxide precursors can be applied using supercycles to create an ASO layer. The aluminum precursor can be TMA. The silicon precursor can be SiCl₄, Tris(tertpentoxy)silanol, diisopropylamino silane (DIPAS) or 1,2-Bis(diisopropylamino)disilane (BDIPADS).

### Coated Particles

In one aspect, the disclosure provides coated particles where a coating is applied to the uncoated particles through the vapor phase deposition method described herein. The coated particles can be coated antagonist particles (particles of one or more antagonists) or coated drug particles (particles of one or more drugs). The coated particles may further contain one or more pharmaceutically acceptable excipients.

The coated particles can have an improved flowability compared to uncoated particles. Applying the coating may improve the wettability and/or dispersibility of the uncoated particles. Applying the coating may improve the dispersibility, but not the wettability of the uncoated particles. Applying the coating may slow the release of the active ingredient in the uncoated particles.

The structure of the antagonist and/or the drug can be assessed by X-Ray Diffraction (XRD) analysis. The structure of antagonist and/or the drug can be assessed by Fourier-transform infrared (FTIR) analysis. The composition of the coated particles can be assessed by Thermogravimetric Analysis (TGA) analysis. The coating process may cause no significant structural change in the antagonist and/or the drug. The dissolution or antagonist/drug release of the coated particles can be assessed by an in vitro release over time (dissolution) analysis. For example, the dissolution or antagonist/drug release of the coated particles can be assessed by HPLC analysis. The dissolution can be assessed by dissolving the coated particles in a sodium phosphate buffer solution (PBS) (e.g., pH 7.2, with or without surfactant) at 37° C., with a stirring of 100 revolutions per minute (RPM), for more than 1 minute, more than 2 minutes, more than 5 minutes, more than 10 minutes, more than 20 minutes, more than 30 minutes, more than 40 minutes, more than 50 minutes, more than 60 minutes, more than 120 minutes, more than 3 hours, more than 4 hours, more than 5 hours, more than 6 hours, more than 7 hours, more than 8 hours, more than 12 hours, more than 16 hours, more than 24 hours. The coated particles can have a reduced dissolution rate compared to uncoated particles. For example, the dissolution rate of the coated particles may be at least more than 5%, more than 10%, more than 20%, more than 30%, more than 40%, more than 50%, more than 60%, more than 70%, more than 80%, more than 90%, more than 100%, more than 110%, more than 120%, more than 130%, more than 140%, more than 150%, more than 200%, more than 300%, more than 400%, more than 500%, or more than 600%, lower than the dissolution of uncoated particles. The uncoated particles may exhibit an immediate release profile.

The morphology of the coated particles can be assessed by Transmission Electron Microscopy (TEM) analysis and/or Scanning Electron Microscopy (SEM) analysis. The coating process may lead to no obvious morphology change.

### Abuse-Deterrent Compositions

In one aspect, the disclosure is related to abuse-deterrent formulations comprising coated antagonist particles. The abuse-deterrent formulation can comprise (1) coated antagonist particles and (2) coated drug particles. The abuse-deterrent formulation can be a dry blend of (1) coated antagonist particles and (2) coated drug particles. The coating in the coated antagonist particles may be different from the coating in the coated drug particles. The coating in the coated antagonist particles may be thicker than the coating in the coated drug particles. Upon tampering (e.g., by crushing or grinding), the coated antagonist particles may release the antagonist. Upon tampering (e.g., by crushing or grinding), the coating in the coated antagonist particles may be damaged, but the coating in the coated drug particles may remain intact.

The abuse-deterrent formulation comprises (1) a coated antagonist particle and (2) a coated drug particle. The coated antagonist particle may consist of an antagonist-containing core and an antagonist coating layer. The coated drug particle may consist of a drug-containing core and a drug coating layer. The antagonist coating layer in the coated antagonist particle may be thicker than the drug coating layer in the coated drug particle. The thickness of the antagonist coating layer in the coated antagonist particle may be above 1 nm, 2 nm, 3 nm, 4 nm, 5 nm, 10 nm, 15 nm, 20 nm, 25 nm, 30 nm, 35 nm, 40 nm, 45 nm, 50 nm, 55 nm, 60 nm, 65 nm, 70 nm, 80 nm, 90 nm, 100 nm, 110 nm, 120 nm, 130 nm, 140 nm, 150 nm, or 200 nm. The thickness of the antagonist coating layer in the coated antagonist particle may be below 1 nm, 2 nm, 3 nm, 4 nm, 5 nm, 10 nm, 15 nm, 20 nm, 25 nm, 30 nm, 35 nm, 40 nm, 45 nm, 50 nm, 55 nm, 60 nm, 65 nm, 70 nm, 80 nm, 90 nm, 100 nm, 110 nm, 120 nm, 130 nm, 140 nm, 150 nm, or 200 nm. The thickness of the antagonist coating layer in the coated antagonist particle may be 50-300 nm, 80-200 nm, or 80-150 nm.

The thickness of the drug coating layer in the coated drug particle may be above 1nm, 2 nm, 3 nm, 4 nm, 5 nm, 10 nm, 15 nm, 20 nm, 25 nm, 30 nm, 35 nm, 40 nm, 45 nm, 50 nm, 55 nm, 60 nm, 65 nm, 70 nm, 80 nm, 90 nm, 100 nm, 110 nm, 120 nm, 130 nm, 140 nm, 150 nm, or 200 nm. The thickness of the drug coating layer in the coated drug particle may be below 1 nm, 2 nm, 3 nm, 4 nm, 5 nm, 10 nm, 15 nm, 20 nm, 25 nm, 30 nm, 35 nm, 40 nm, 45 nm, 50 nm, 55 nm, 60 nm, 65 nm, 70 nm, 80 nm, 90 nm, 100 nm, 110 nm, 120 nm, 130 nm, 140 nm, 150 nm, or 200 nm. The thickness of the drug coating layer in the coated drug particle may be 5-100 nm, 10-50 nm, or 10-30 nm.

Both the antagonist coating layer and the drug coating layer can be prepared by supercycles as described herein. Both the antagonist coating layer and the drug coating layer can comprise an AZO coating. The Al/Zn ratio of the antagonist coating layer may be higher than the Al/Zn ratio of the drug coating layer. The Al/Zn ratio (wt/wt) of the antagonist coating layer in the coated antagonist particle may be more than 0.01, more than 0.02, more than 0.03, more than 0.04, more than 0.05, more than 0.06, more than 0.07, more than 0.08, more than 0.09, more than 0.1, more than 0.11, more than 0.12, more than 0.13, more than 0.14, more than 0.15, more than 0.2, more than 0.25, more than 0.3, more than 0.35, more than 0.4, more than 0.45, more than 0.5, more than 0.6, more than 0.7, more than 0.9, or more than 0.9. The Al/Zn ratio of the antagonist coating layer in the coated antagonist particle may be less than 0.01, less than 0.02, less than 0.03, less than 0.04, less than 0.05, less than 0.06, less than 0.07, less than 0.08, less than 0.09, less than 0.1, less than 0.11, less than 0.12, less than 0.13, less than 0.14, less than 0.15, less than 0.2, less than 0.25, less than 0.3, less than 0.35, less than 0.4, less than 0.45, less than 0.5, less than 0.6, less than 0.7, less than 0.9, or less than 0.9. The Al/Zn ratio of the antagonist coating layer in the coated antagonist particle may be 0.1-0.5, 0.1-0.4, 0.1-0.35, 0.15-0.35 or 0.25-0.35.

The Al/Zn ratio (wt/wt) of the drug coating layer in the coated drug particle may be more than 0.01, more than 0.02, more than 0.03, more than 0.04, more than 0.05, more than 0.06, more than 0.07, more than 0.08, more than 0.09, more than 0.1, more than 0.11, more than 0.12, more than 0.13, more than 0.14, more than 0.15, more than 0.2, more than 0.25, more than 0.3, more than 0.35, more than 0.4, more than 0.45, more than 0.5, more than 0.6, more than 0.7, more than 0.9, or more than 0.9. The Al/Zn ratio of the drug coating layer in the coated drug particle may be less than 0.01, less than 0.02, less than 0.03, less than 0.04, less than 0.05, less than 0.06, less than 0.07, less than 0.08, less than 0.09, less than 0.1, less than 0.11, less than 0.12, less than 0.13, less than 0.14, less than 0.15, less than 0.2, less than 0.25, less than 0.3, less than 0.35, less than 0.4, less than 0.45, less than 0.5, less than 0.6, less than 0.7, less than 0.9, or less than 0.9. The Al/Zn ratio of the drug coating layer in the coated drug particle may be 0.3-0.9, 0.3-0.7, 0.4-0.7, or 0.4-0.6.

Upon normal oral ingestion, the drug release rate of the coated drug particle can be faster than the antagonist release rate of the coated antagonist particle. Upon normal oral ingestion, the drug in the coated drug particle can be released while the antagonist in the coated antagonist particle is not released. Upon tampering (e.g., grinding), the antagonist coating in the coated antagonist particle may be destroyed and the antagonist is released.

When the abuse-deterrent formulation is tampered with (e.g., by crushing or grinding), the antagonist can be released, thereby preventing the drug from having its intended effect. When the abuse-deterrent formulation is not tampered with, the majority of the antagonist is not released. Tampering with the abuse-deterrent formulation can increase the release of the antagonist by more than 10%, 10%, 20%, 30%, 40%, 50%, 60%, 70%, 80%, 90%, 100%, 200%, 300%, 400%, 500%, 1000%, 2000%, 5000%, or 10,000%. Tampering with the abuse-deterrent formulation can increase the release of the antagonist by less than 10%, 10%, 20%, 30%, 40%, 50%, 60%, 70%, 80%, 90%, 100%, 200%, 300%, 400%, 500%, 1000%, 2000%, 5000%, or 10,000%.

The ratio between (1) coated antagonist particles and (2) coated drug particles can be more than 0.0.1, 0.02, 0.05, 0.1, 0.2. 0.3. 0.4, 0.5, 0.6, 0.7, 0.8, 0.9, 1, 1.5, 2, 3, 4, 5, 6, 7, 8, 9, 10, 20, 50, or 100. The ratio between (1) coated antagonist particles and (2) coated drug particles can be less than 0.0.1, 0.02, 0.05, 0.1, 0.2. 0.3. 0.4, 0.5, 0.6, 0.7, 0.8, 0.9, 1, 1.5, 2, 3, 4, 5, 6, 7, 8, 9, 10, 20, 50, or 100. The coated antagonist particles may constitute more than 1%, 2%, 3%, 4%, 5%, 6%, 7%, 8%, 9%, 10%, 20%, 30%, 40%, 50%, 60%, 70%, 80%, 90% of the abuse-deterrent formulation by weight. The coated antagonist particles may constitute less than 1%, 2%, 3%, 4%, 5%, 6%, 7%, 8%, 9%, 10%, 20%, 30%, 40%, 50%, 60%, 70%, 80%, 90% of the abuse-deterrent formulation by weight. The coated drug particles may constitute more than 1%, 2%, 3%, 4%, 5%, 6%, 7%, 8%, 9%, 10%, 20%, 30%, 40%, 50%, 60%, 70%, 80%, 90% of the abuse-deterrent formulation by weight. The coated drug particles may constitute less than 1%, 2%, 3%, 4%, 5%, 6%, 7%, 8%, 9%, 10%, 20%, 30%, 40%, 50%, 60%, 70%, 80%, 90% of the abuse-deterrent formulation by weight.

The pharmaceutical compositions can be formulated in any suitable manner known in the art. For example, the pharmaceutical compositions can be in the form of tablets, capsules, powders, microparticles, granules, syrups, suspensions, solutions, nasal spray, transdermal patches, injectable solutions, or suppositories.

Pharmaceutical compositions are formulated to be compatible with their intended route of administration (e.g., oral, intravenous, intraarterial, intramuscular, intradermal, subcutaneous, or intraperitoneal). The compositions can include a sterile diluent (e.g., sterile water or saline), a fixed oil, polyethylene glycol, glycerine, propylene glycol or other synthetic solvents, antibacterial or antifungal agents (e.g., benzyl alcohol or methyl parabens, chlorobutanol, phenol, ascorbic acid, and thimerosal), antioxidants (e.g., ascorbic acid and sodium bisulfite), chelating agents (e.g., ethylenediaminetetraacetic acid), buffers (e.g., acetates, citrates, and phosphates), and isotonic agents (e.g., sugars (e.g., dextrose), polyalcohols (e.g., mannitol or sorbitol), and salts (e.g., sodium chloride)), or any combination thereof. Liposomal suspensions can also be used as pharmaceutically acceptable carriers (see, e.g., U.S. Pat. No. 4,522,811). Preparations of the compositions can be formulated and enclosed in ampules, disposable syringes, or multiple dose vials. Where required (as in, for example, injectable formulations), proper fluidity can be maintained by, for example, the use of a coating (e.g., lecithin) or a surfactant. Controlled release can be achieved by implants and microencapsulated delivery systems, which can include biodegradable, biocompatible polymers (e.g., ethylene vinyl acetate, polyanhydrides, polyglycolic acid, collagen, polyorthoesters, and polylactic acid).

Pharmaceutically acceptable carriers, adjuvants and vehicles that can be used in the pharmaceutical compositions of the present disclosure include ion exchangers, alumina, aluminum stearate, lecithin, serum proteins (e.g., human serum albumin), buffer substances (e.g., phosphates, glycine), sorbic acid, potassium sorbate, partial glyceride mixtures of saturated vegetable fatty acids, water, salts or electrolytes (e.g., protamine sulfate, disodium hydrogen phosphate, potassium hydrogen phosphate, sodium chloride, and zinc salts), colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol, and wool fat.

The compositions or formulations can contain the coated particles described herein in the range of 0.001% to 100% (e.g., 0.1-95%, 20-80%, or 75-85%) with the balance made up from the suitable pharmaceutically acceptable excipients.

The coating may simply the formulation process or other manufacturing process of a pharmaceutical composition. For example, the coating may eliminate the need to include additional detergents in the final formulation.

### EXAMPLES

### Example 1: The Normal Use of an Abuse-Deterrent Formulation (ADF)

An exemplary abuse-deterrent formulation is prepared by mixing coated opioid particles and coated opioid antagonist particles. The coated opioid particles and the coated opioid antagonist particles have different coatings.

FIG. 6 depicts a schematic illustration of the normal use (administered as prescribed) of a drug formulation comprising coated opioid particles and coated opioid antagonist particles. The coated opioid particles and coated opioid antagonist particles are mixed together inside of a capsule. When the capsule is ingested normally by a patient, the opioid is released (therapeutic window), while the opioid antagonist is not released or released in a minimal amount (outside the therapeutic window).

### Example 2: Tampering of an ADF by Grinding

An exemplary abuse-deterrent formulation is prepared by mixing coated opioid particles and coated opioid antagonist particles. The coated opioid particles and the coated opioid antagonist particles have different coatings.

FIG. 7 depicts a schematic illustration of the tampering (grinding) of a drug formulation comprising coated opioid particles and coated opioid antagonist particles. The coated opioid particles and coated opioid antagonist particles are mixed together inside of a capsule. When the capsule is tampered by grinding (e.g., crushed using a mortar and pestle), both the opioid and the opioid antagonist are released, destroying the therapeutic effects.

### Example 3: Tampering of an ADF by Solvent Dissolution

An exemplary abuse-deterrent formulation is prepared by mixing coated opioid particles and coated opioid antagonist particles. The coated opioid particles and the coated opioid antagonist particles have different coatings.

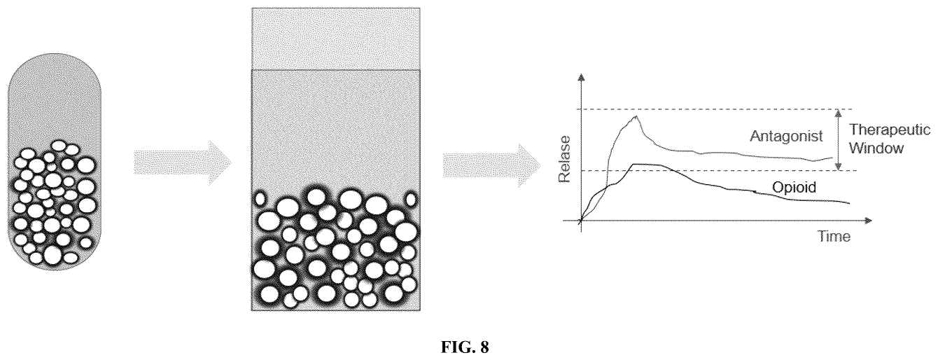

FIG. 8 depicts a schematic illustration of the tampering (solvent dissolution) of a drug formulation comprising coated opioid particles and coated opioid antagonist particles. The coated opioid particles and coated opioid antagonist particles are mixed together inside of a capsule. When the capsule is tampered by solvent dissolution (e.g., using lemonade or vinegar), both the opioid and the opioid antagonist are released, destroying the therapeutic effects.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. An abuse-deterrent pharmaceutical composition, comprising:
a drug particle comprising a drug; and
a coated antagonist particle comprising (a) an antagonist containing core comprising an antagonist to the drug, and (b) an antagonist coating layer enclosing the antagonist-containing core, wherein the antagonist coating layer comprises an inorganic oxide that comprises aluminum, zinc, silicon, and/or titanium.

2. The abuse-deterrent pharmaceutical composition of claim 1, wherein the antagonist coating layer reduces the release rate of the antagonist but does not reduce the release rate of the drug.

3. The abuse-deterrent pharmaceutical composition of claim 1, wherein the drug particle comprises a drug-containing core comprising a drug and a drug coating layer enclosing the drug-containing core, wherein the drug coating layer comprises an inorganic oxide that comprises aluminum, zinc, silicon, and/or titanium selected from the group consisting of aluminum oxide, zinc oxide, silicon oxide and titanium oxide.

4. The abuse-deterrent pharmaceutical composition of claim 2, wherein the antagonist coating layer in the coated antagonist particle is different from the drug coating layer in the coated drug particle.

5.-11. (Cancelled)

12. The abuse-deterrent pharmaceutical composition of claim 1, wherein the antagonist is selected from the group consisting of naltrexone, naloxone, nalmefene, cyclazacine, levallorphan, pharmaceutically acceptable salts thereof, and combinations thereof.

13. The abuse-deterrent pharmaceutical composition of claim 1, wherein the drug is an opioid.

14. The abuse-deterrent pharmaceutical composition of claim 1, wherein the inorganic oxide is selected from the group consisting of aluminum oxide, zinc oxide, silicon oxide and titanium oxide.

15. The abuse-deterrent pharmaceutical composition of claim 1, wherein the molar ratio between (1) the antagonist in the composition and (2) the drug in the composition is above 1.

16. The abuse-deterrent pharmaceutical composition of claim 1,wherein the molar ratio between (1) the antagonist in the composition and (2) the drug in the composition is above 2.

17. The abuse-deterrent pharmaceutical composition of claim 1, wherein both the antagonist coating layer and the drug coating layer comprise aluminum and zinc, and the aluminum/zinc ratio in the antagonist coating layer is higher than the aluminum/zinc ratio in the drug coating layer.

18. The abuse-deterrent pharmaceutical composition of claim 1, comprising a dry mix of the coated antagonist particle and the optionally coated drug particle.

19. (canceled)

20. The abuse-deterrent pharmaceutical composition of claim 1, wherein the antagonist negates the intended effect of the drug or produces an unpleasant or punishing stimulus or effect.

21. The abuse-deterrent pharmaceutical composition of claim 1, wherein the drug is an opioid, and when the particles are administered in their native state (without crushing), the plasma level of the antagonist is insufficient to blunt the effects of the opioid.

22. The abuse-deterrent pharmaceutical composition of claim 21, wherein the drug is an opioid, and the when the particles are crushed and administered, the plasma level of the antagonist is sufficient to blunt the effects of the opioid.

23. The abuse-deterrent pharmaceutical composition of claim 1, wherein the drug is an opioid, and the when the particles are tampered by solvent dissolution (e.g., using an acidic solution such as lemonade or vinegar) and administered, the plasma level of the antagonist is sufficient to blunt the effects of the opioid.

24. The abuse-deterrent pharmaceutical composition of claim 1, wherein, upon tampering (e.g., grinding), the dissolution rate of the coated antagonist particle is increased.

25. (canceled)

26. A method of preparing an abuse-deterrent pharmaceutical composition, the method comprising the sequential steps of:
(a) loading particles comprising an antagonist into a chamber of a reactor;
(b1) applying a vaporous or gaseous precursor to the particles in the reactor by pulsing the vaporous or gaseous aluminum precursor into the reactor;
(b2) performing one or more pump-purge cycles using an inert gas;
(b3) applying a vaporous or gaseous oxidant to the particles in the reactor by pulsing the oxidant into the reactor;
(b4) performing one or more pump-purge cycles using an inert gas;
(c) repeating steps (b1)-(b4) at least once to create coated antagonist particles; and
(d) mixing the coated antagonist particles with coated drug particles comprising a drug, wherein the antagonist is an antagonist to the drug.

27. (canceled)

28. The method of claim 26, wherein the coated drug particles are prepared by a method comprising the sequential steps of:
(A) loading particles comprising a drug-containing core comprising a drug into a chamber of a reactor;
(B1) applying a vaporous or gaseous precursor to the particles in the reactor by pulsing the vaporous or gaseous aluminum precursor into the reactor;
(B2) performing one or more pump-purge cycles using an inert gas;
(B3) applying a vaporous or gaseous oxidant to the particles in the reactor by pulsing the oxidant into the reactor;
(B4) performing one or more pump-purge cycles using an inert gas;
(C) repeating steps (B1)-(B4) at least once to create the coated drug particles.

29.-41. (canceled)

42. A method of preparing an abuse-deterrent pharmaceutical composition, the method comprising the sequential steps of:
(a) loading particles comprising an antagonist into a chamber of a reactor;
(b) performing a first number of first cycles, wherein each first cycle comprises steps (b1)-(b4):
(b1) applying a vaporous or gaseous first inorganic oxide precursor to the particles in the reactor by pulsing the vaporous or gaseous first inorganic oxide precursor into the reactor;
(b2) performing one or more pump-purge cycles of the reactor using an inert gas;
(b3) applying a vaporous or gaseous oxidant to the particles in the reactor by pulsing the oxidant into the reactor;
(b4) performing one or more pump-purge cycles of the reactor using an inert gas;
(c) performing a second number of second cycles, wherein each second cycle comprises steps (c1)-(c4):
(c1) applying a vaporous or gaseous second inorganic oxide precursor to the particles in the reactor by pulsing the vaporous or gaseous second inorganic oxide precursor into the reactor;
(c2) performing one or more pump-purge cycles of the reactor using an inert gas;
(c3) applying a vaporous or gaseous oxidant to the particles in the reactor by pulsing the oxidant into the reactor;
(c4) performing one or more pump-purge cycles of the reactor using an inert gas; and
(d) repeating steps (b)-(c) at least once to create coated antagonist particles; and
(e) mixing the coated antagonist particles with coated drug particles comprising a drug,
wherein the antagonist is an antagonist to the drug,
wherein, the first inorganic oxide precursor and second inorganic oxide precursor are different and the first number is an integer selected from 1-10, and the second number is an integer selected from 1-10.

43. (canceled)

44. The method of claim 42, wherein the coated drug particles are prepared by a method comprising the sequential steps of:
(A) loading a drug-containing core comprising a drug into a chamber of a reactor;
(B) performing a first number of first cycles, wherein each first cycle comprises steps (B1)-(B4):
(B1) applying a vaporous or gaseous first inorganic oxide precursor to the particles in the reactor by pulsing the vaporous or gaseous first inorganic oxide precursor into the reactor;
(B2) performing one or more pump-purge cycles of the reactor using an inert gas;
(B3) applying a vaporous or gaseous oxidant to the particles in the reactor by pulsing the oxidant into the reactor;
(B4) performing one or more pump-purge cycles of the reactor using an inert gas; (C) performing a second number of second cycles, wherein each second cycle comprises steps (C1)-(C4):
(C1) applying a vaporous or gaseous second inorganic oxide precursor to the particles in the reactor by pulsing the vaporous or gaseous second inorganic oxide precursor into the reactor;
(C2) performing one or more pump-purge cycles of the reactor using an inert gas;
(C3) applying a vaporous or gaseous oxidant to the particles in the reactor by pulsing the oxidant into the reactor;
(C4) performing one or more pump-purge cycles of the reactor using an inert gas; and (D) repeating steps (B)-(C) at least once to create coated drug particles,
wherein, the first inorganic oxide precursor and second inorganic oxide precursor are different and the first number is an integer selected from 1-10, and the second number is an integer selected from 1-10.

45.-77. (canceled)
